(19) United States
(12) Patent Application Publication
(10) Pub. No.: US 2010/0184812 A1
(43) Pub. Date: Jul. 22, 2010

(54) MUTUAL PRODRUGS AND METHODS TO TREAT CANCER

(75) Inventors: Vincent C. O. Njar, Glen Burnie, MD (US); Lalji K. Gediya, Secane, PA (US); Aakanksha Khandelwal, Silver Spring, MD (US)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: UNIVERSITY OF MARYLAND, BALTIMORE, Baltimore, MD (US)

(21) Appl. No.: 12/663,194

(22) PCT Filed: Jun. 6, 2008
(86) PCT No.: PCT/US08/66103

§ 371 Date: Dec. 4, 2009

Related U.S. Application Data
(60) Provisional application No. 60/924,932, filed on Jun. 6, 2007.
(60) Provisional application No. 60/924,995, filed on Jun. 7, 2007.

(51) Int. Cl. A61K 31/4406; C07C 271/62; C07D 213/56; C07C 271/28; A61K 31/27; A61P 35/00; A61P 35/02

(52) U.S. Cl. 514/357; 554/106; 546/335; 514/487

(57) ABSTRACT

Mutual prodrugs comprising retinoids and histone deacetylase inhibitors, methods for production of the mutual prodrugs, and methods of treatment comprising administration of the mutual prodrugs. The retinoids include all-trans retinoic acid, 13-cis retinoic acid, and retinoic acid analogs that have a substitution at C-4. Further, the mutual prodrugs of the present invention can be used as therapeutic agents for the treatment of cancer and dermatological diseases and conditions. Pharmaceutical compositions comprising the mutual prodrugs.

### CROSS REFERENCE TO RELATED APPLICATIONS

This application claims the benefit of U.S. Provisional Application 60/924,932 filed Jun. 6, 2007 and U.S. Provisional Application 60/924,995 filed Jun. 7, 2007, both of which are hereby incorporated by reference.

### STATEMENT REGARDING FEDERALLY SPONSORED RESEARCH OR DEVELOPMENT

The invention was made with support of grants from the U.S. Department of Defense, Grant Nos: DAMD17-01-1-0549 and W81XWH-04-1-0101; and National Institutes of Health, Grant No: 1R21CA117991. The U.S. Government has certain rights in this invention.

### BACKGROUND OF INVENTION

1. Field of Invention

The invention relates to mutual prodrugs comprising retinoids and histone deacetylase inhibitors, methods for production of the mutual prodrugs, and methods of treatment comprising administration of the mutual prodrugs to subjects in need of treatment. The retinoids include all-trans retinoic acid, 13-cis retinoic acid, retinoic acid analogs that have a substitution at C-4 (hereafter referred to as C-4 substituted retinoic acid analogs except otherwise stated) and a retinoic acid metabolism blocking agents (RAMBAs). The mutual prodrugs of the present invention can be used as therapeutic agents for the treatment of cancer and dermatological diseases and conditions. This invention also relates to pharmaceutical compositions comprising the mutual prodrugs.

2. Description of the Related Art

Prostate cancer (PCA) is the most common malignancy and age-related cause of cancer death worldwide. Apart from lung cancer, PCA is the most common form of cancer in men and the second leading cause of death in American men. In the United States in 2004, an estimated 230,000 new case of prostate cancer were diagnosed and about 23,000 men died of this disease (Jemal A et al., CA Cancer J. Clin., 54: 8-29, 2004). During the period of 1992 to 1999, the average annual incidence of PCA among African American men was 59% higher than among Caucasian men, and the average annual death rate was more than twice that of Caucasian men (American Cancer Society—Cancer Facts and Figures 2005).

The growth of most prostate tumors depends largely on exposure to androgens during the initial stages of tumor development, and thus, anti-hormonal therapy, by surgical or medical suppression of androgen action, remains a major treatment option of the disease (Denmeade S R and Isaacs J T, Nature Rev. Cancer 2: 389-396, 2002). Although this treatment may be initially successful, most tumors eventually recur due to the expansion of an androgen-refractory population of PCA cells (Oesterling J et al., Cancer of the prostate, In: Cancer: Principles and Practice of Oncology, 5^{th} ed., DeVita V T et al. (eds), pp. 1322-1386, Philadelphia: Lippincott-Raven Publishers, 1997). Metastatic disease that develops even after potentially curative surgery remains a major clinical challenge. Therapeutic treatments for patients with metastatic PCA are limited because current chemotherapeutic and radiotherapeutic regimens are largely ineffective (Feldman B J and Feldman D, Nat. Rev. Cancer, 1: 34-45, 2001). Hence, there is urgent need to develop new therapeutic agents with defined targets to prevent and treat this disease.

PCA tumors that arise after anti-hormonal therapy generally are less differentiated and it is believed that agents that can induce the cells to differentiate would represent a new therapeutic strategy (Sartorreli A C, Br. J. Cancer, 52: 293-302, 1985). Hence, the goal of differentiation therapy is to induce malignant cells to pass the block to maturation, thereby allowing them to progress to more differentiated cell types with less proliferative ability.

### HDACIs

Histone Deacetylases (HDACs) are the catalytic subunits of multiprotein complexes responsible for deacetylation of histones and nonhistone proteins. Lysine acetylation, i.e., the transfer of an acetyl moiety from acetyl-coenzyme A to the ε-amino group of a specific lysine residue, has emerged as the major form of posttranslational modification of histones, and other proteins have been correlated with transcription, chromatin assembly, DNA repair, and recombinatorial events (Marks et al., Histone deacetylases and cancer. causes and therapies, Nat Rev Cancer, 1: 194-202, 2001.). Histone acetylation in vivo is a dynamic, reversible process governed by the opposite actions of histone acetyltransferases (HATs) and HDACs. Aberrant acetylation of histone tails, emerging from either HAT mutation or abnormal recruitment of HDACs, has been linked to carcinogenesis (Pandolfi, P. P. Transcription therapy for cancer, Oncogene, 20: 3116-3127, 2001). In various cases, altered HAT or HDAC activity has been identified in a variety of cancers. It has recently been demonstrated that the expression and activity of HDAC1 is up-regulated in prostate cancer compared to benign prostatic hyperplasia (BPH) (Patra et al., Histone deacetylase and DNA methyltransferase in human prostate cancer, Biochem Biophys Res Commun, 287: 705-713, 2001).

HDACIs have been found to be useful for the activation of genes responsive to hormone receptors. HDACIs are potent inducers of growth arrest, differentiation and/or apoptosis of several cell lines, and they constitute a novel class of chemotherapeutic agents initially identified by their ability to reverse the malignant phenotype of transformed cells. They have been shown to activate differentiation programs, inhibit cell cycle, and induce apoptosis in a wide range of tumor-derived cell lines and to block angiogenesis and stimulate the immune system in vivo (Marks et al., Histone deacetylases and cancer, causes and therapies, Nat Rev Cancer, 1: 194-202, 2001; Johnstone, R. W. Histone-deacetylase inhibitors, novel drugs for the treatment of cancer, Nat Rev Drug Discov, 1: 287-299, 2002.). Whereas the mechanisms through which HDACIs exert these anti-tumor activities have not been fully delineated, induction of histone hyperacetylation and modulation of gene transcription through chromatin remodeling are thought to be primarily responsible, leading to the selective activation of genes associated with cell growth and survival. Suberoylanilide hydroxamic acid (SAHA, VORINOSTAT® or ZOLINZA®) was approved in 2006 for the treatment of patients with relapsed or refractory cutaneous T-cell lymphoma (Marks et al, Dimethyl sulfoxide to vorinostat, development of this histone deacetylase inhibitor as an anticancer drug, Nat Biotechnol, 25: 84-90, 2007.).

One of the early HDACIs discovered is N-hydroxy-N¹-phenylactanediamide, also called suberanilide hydroxamic acid (SAHA). (Richon et al., Second generation hybrid polar compounds are potent inducers of transformed cell differentiation, Proc Natl Acad Sci U S A 1996, 93, (12), 5705-8; Kelly et al., Phase I clinical trial of histone deacetylase inhibitor, suberoylanilide hydroxamic acid administered intravenously, Clin Cancer Res 2003, 9, (10 Pt 1), 3578-88.) This compound (trade name: Vorinostat®) was approved in 2006 by the U.S. Food and Drug Administration (FDA) for the treatment of advanced cutaneous T-cell-lymphoma. (Bolden et al., Anticancer activities of histone deacetylase inhibitors, Nat Rev Drug Discov 2006, 5, (9), 769-84.)

Histone acetyltransferase and histone deacetylase (HDAC) have opposing effect on transcription (Ito et al., Histone acetylation and histone deacetylation, Mol Biotechnol, 20: 99-106, 2002; Kuo et al., Roles of histone acetyltransferases and deacetylases in gene regulation, Bioessays, 20: 615-626, 1998.). Often, DNA methylation and histone deacetylation of tumor suppressor genes occur in many human cancers, leading to suppression of function of these genes thereby conferring a growth advantage for the tumor cells (Macaluso et al., A. How does DNA methylation mark the fate of cells?, Tumori, 90: 367-372, 2004; Robertson et al. DNA methylation: past, present and future directions, Carcinogenesis, 21: 461-467, 2000.). It has recently been demonstrated that the expression and activity of HDAC1 is up-regulated (2-4-fold) in prostate cancer compared to benign prostatic hyperplasia (Patra et al., Histone deacetylase and DNA methyltransferase in human prostate cancer, Biochem Biophys Res Commun, 287: 705-713, 2001.). HDACIs, such as SAHA, and N-(2-aminophenyl)4-[N-(pyridine-3-yl-methoxy-carbonyl)aminomethyl]benzamide (MS-275) can directly interact with the HDAC enzymes at the catalytic site and inhibit their function (Bolden et al, Anticancer activities of histone deacetylase inhibitors, Nat Rev Drug Discov, 5: 769-784, 2006; Marks et al., Histone deacetylase inhibitors: inducers of differentiation or apoptosis of transformed cells, J Natl Cancer Inst, 92: 1210-1216, 2000; Minucci et al., Histone deacetylase inhibitors and the promise of epigenetic (and more) treatments for cancer, Nat Rev Cancer, 6: 38-51, 2006.). This leads to acetylation of histones which opens-up the chromatin structure allowing transcription of anti-growth and pro-apoptotic genes to occur. MS-275 is now in phase I/II clinical trials for various solid tumors and hematological malignancies (Hess-Stumpp et al., MS-275, a potent orally available inhibitor of histone deacetylases—The development of an anticancer agent, Int J Biochem Cell Biol, 39: 1388-1405, 2007.).

Suberoylanilide hydroxamic acid (SAHA) is a modest HDACI and has been used extensively in vitro and in vivo in cancer models (Butler et al., Suberoylanilide hydroxamic acid, an inhibitor of histone deacetylase, suppresses the growth of prostate cancer cells in vitro and in vivo, Cancer Res, 60: 5165-5170, 2000.) and it is the only HDACI currently approved for clinical use (Marks et al, Dimethyl sulfoxide to vorinostat, development of this histone deacetylase inhibitor as an anticancer drug, Nat Biotechnol, 25: 84-90, 2007; Richon et al., Histone deacetylase inhibitors, development of suberoylanilide hydroxamic acid (SAHA) for the treatment of cancers, Blood Cells Mol Dis, 27: 260-264, 2001.). The HDACI benzamide compound MS-275 is in several clinical trials as a potential therapy for a variety of cancers (Hess-Stumpp et al, MS-275, a potent orally available inhibitor of histone deacetylases—the development of an anticancer agent, Int J Biochem Cell Biol, 39: 1388-1405, 2007.).

Retinoids

Retinoids, including retinoic acids (RAs), are also thought to play a role in cellular differentiation. All-trans retinoic acid (ATRA), the most biologically active metabolite of vitamin A, is known to play a major role in cellular differentiation and proliferation of epithelial tissues.

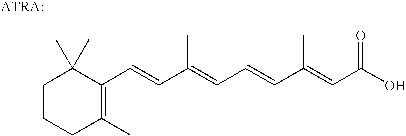

When added exogenously, ATRA has been shown to have the ability to redirect cells towards their normal phenotype, and as such, may be useful in reversing or suppressing evolving malignant lesions or in preventing cancer invasion (Hill D L and Grubbs C J, Annu Rev Nutr 12: 161-181, 1992; Hong W K and Itri L, Retinoids and human cancer, In The Retinoids: Biology, Chemistry and Medicine, Sporn M B et al. (eds), pp 597-630, Raven Press: New York, 1994). In addition, ATRA is known to have therapeutic effects in the treatment of many dermatological diseases.

The ability to effectively use retinoids such as ATRA may be compromised due to the rapid in vivo catabolism by cytochrome P450-dependent enzymes (Muindi J et al., Blood 79: 299-303, 1992; Smith M A et al., J Clin Oncol 10: 839-864, 1992; Warrell R P Jr., Differentiating agents, In Cancer, principles and practice of oncology; DeVita Jr et al. (eds), Vol. I, pp 483-490, Lippincott: Philadelphia, 1997; Kizaki M et al., Blood, 87: 725-733, 1996; Cunliffe, Biochem Soc Trans, 14: 943-945, 1986; Griffiths C E M et al., Br Journal of Dermatology, 127 (suppl):21-24, 1992)). Indeed, ATRA can be metabolized through several routes. The physiologically most prominent pathway starts with hydroxylation at the 4-position of the cyclohexenyl ring, leading to the formation of 4-hydroxy-ATRA that is then converted to more polar metabolites via 4-oxo-ATRA (Frolik C A et al., Biochemistry 18: 2092-2097, 1979; Frolik C A et al. J Biol Chem 255: 8057-8062, 1980; Roberts A B et al., J Biol Chem 254: 6296-6302, 1979; Roberts A B et al., Arch Biochem Biophys 199: 374-383, 1980; Van Wauwe J et al., Biochem Pharmacol 47: 737-741, 1994; Napoli J L, FASEB J 10: 993-1001, 1996).

The first and third catabolic steps are catalyzed by a cytochrome P450-dependent enzyme complex (Frolik C A et al., J Biol Chem 255: 8057-8062, 1980; Leo M A et al., Arch Biochem Biophys 243: 305-312, 1984; Van Heusden J et al., Br J Cancer 77: 26-32, 1998; Van Heusden J et al., Br J Cancer 77: 1229-1235, 1998). Although the exact nature of this enzyme remains to be elucidated, a cytochrome P450 enzyme (designated CYP26) with specific ATRA 4-hydroxylase activity, which is also rapidly induced by ATRA, has recently been cloned from zebra fish, mouse and man (Hague M et al., Nutri Rev 56: 84-85, 1999; Sonneveld E and Vander Sagg P T, Inter. J Vit Nutr Res 68: 404-410, 1998).

In principle, inhibitors of ATRA 4-hydroxylase should increase endogenous levels of ATRA (acting as ‘ATRA-mimetics’) and overcome some ATRA-resistance. A number of azole compounds that are known to inhibit particular cytochrome P450 enzymes have also been shown to be inhibitors of ATRA 4-hydroxylase (Williams J B and Napoli J L, PNAS USA 82: 4658-4662, 1985; Williams J B and Napoli J L, Biochem Pharmacol 36: 1386-1388, 1987; Napoli J L, Retinoic acid biosynthesis and metabolism, FASEB J 10: 993-1001, 1996; Roberts A B et al., J Biol Chem 254: 6296-6302, 1979; Vanden Bossche H et al., Skin Pharmacology 1: 176-185, 1988; Van Wauwe J P et al., J Pharmacol Exp Ther, 245:718-722, 1988; Freyne E et al., Bioorg Med Chem Lett 8: 267-272, 1998).

Specific inhibitors of retinoic acid metabolism, termed retinoic acid metabolism blocking agents or “RAMBAs”, have been identified (Patel, J B et al., J. Med. Chem.47: 6716-6729, 2004; Belosay, A et al., Cancer Res. 66: 11485-11693, 2006; Patel J B et al., Br. J. Cancer, 96: 1204-1215, 2007). These compounds are able to enhance the anti-proliferative effects of ATRA in breast and prostate cancer cells in vitro (Huynh C K et al., Br. J. Cancer, 94: 513-523, 2006). In addition, RAMBAs have been shown to induce differentiation and apoptosis in these cancer cell lines, with the breast cancer cell lines more sensitive to the effects of RAMBAs. The discovery of such agents has led to interest in using RAMBAs in the treatments of cancer (see, e.g., Miller, Jr., W. H., Cancer, 83, 1471-1482, 1998; Njar V C O et al., Bioorg. Med. Chem, 14: 4323-4340, 2006; Njar V C O et al., Med. Chem., 2: 431-438, 2006).

### BRIEF SUMMARY OF THE INVENTION

The present invention is includes compounds, compositions, manufacturing methods thereof, as well as uses thereof in treatment and/or prevention.

The present invention includes a mutual prodrug compound comprising (i) one or more HDACIs linked to (ii) one or more retinoids.

The present invention also includes a mutual prodrug compound comprising (i) one or more HDACIs linked to (ii) one or more RAMBAs.

The present invention relates to mutual prodrugs where the components are chemically linked, that is, where one or more HDACIs is chemically linked to one or more retinoids, one or more HDACIs is chemically linked to one or more RAMBAs, or one or more HDACIs is chemically linked to both one or more retinoids and one or more RAMBAs. A mutual prodrug (hybrid drug) is a type of carrier linked prodrug where the carrier used is another pharmacologically active compound instead of some inert molecule. Typically, when two synergistic agents are administered individually but simultaneously, they will be transported to the site of action with different efficiencies. However, when it is desirable to have the two agents reach a site simultaneously, the MP strategy may be used to an advantage. The MP of ATRA and butyric acid (BA) called retinoyloxymethyl butyrate, 3 (RN1) has been shown to function at lower concentrations than ATRA or BA alone in the ATRA-sensitive leukemia cell line HL-60 (Nudelman, A.; Rephaeli, A., Novel mutual prodrug of retinoic and butyric acids with enhanced anticancer activity. J Med Chem 2000, 43, 2962-2966). RN1 was found to exhibit significant growth inhibitory activity in both ATRA-sensitive and -resistant APL cells (Maim, K. K.; Rephaeli, A.; Colosimo, A. L.; Diaz, Z.; Nudelman, A.; Levovich, I.; Jing, Y.; Waxman, S.; Miller, W. H., Jr., A retinoid/butyric acid prodrug overcomes retinoic acid resistance in leukemias by induction of apoptosis. Mol Cancer Res 2003, 1, 903-912).

Applicants have designed and synthesized novel MPs of retinoic acids and HDIs. The compounds of the invention were evaluated against several breast (MCF-7, MCF-7_{TAMR}, MCF-7_{HOXB7} LTLC, LTLT-Ca, and MDA-MB-231) and prostate (PC-3) cancer cell lines, most of which are generally resistant to most therapeutic agents, and were found to be potent anti-neoplastic agents. Importantly, the MPs possess enhanced anticancer activities compared to the parent compounds or their combinations.

The mutual prodrug compounds may be used in vitro or in vivo to inhibit growth of a cell; in vitro or in vivo to inhibit growth of a cancer cell; or in vitro or in vivo to treat a subject in need thereof.

The mutual prodrug compounds may be formulated in pharmaceutically acceptable mutual prodrug compositions, comprising one or more of the mutual prodrug compounds of the present invention and a pharmaceutically acceptable carrier or an excipient.

The present invention includes methods of inhibiting the activity of ATRA 4-hydroxylase using one or more of the mutual prodrug compounds of the present invention. In particular, the present invention includes methods of inhibiting the activity of ATRA 4-hydroxylase in a subject comprising administering one or more of the mutual prodrug compounds of the present invention to a subject. In a preferred embodiment, the present invention includes methods of inhibiting the activity of ATRA 4-hydroxylase in a subject comprising administering one or more of the pharmaceutically-acceptable mutual prodrug compounds of the present invention to a subject.

The present invention includes methods of inhibiting the growth of a cell using one or more of the mutual prodrug compounds of the present invention. In particular, the present invention includes methods of inhibiting the growth of a cancer cell in a subject comprising administering one or more of the mutual prodrug compounds of the present invention. In a preferred embodiment, the present invention includes methods of inhibiting the growth of a cancer cell in a subject comprising administering one or more of the pharmaceutically-acceptable mutual prodrug compounds of the present invention to a subject.

The present invention includes methods of treating cancer using one or more of the mutual prodrug compounds of the present invention. In particular, the present invention includes methods of treating cancer in a subject in need of treatment, comprising administering one or more of the mutual prodrug compounds of the present invention to a subject. In a preferred embodiment, the present invention includes methods of treating cancer in a subject in need of treatment, comprising administering one or more of the pharmaceutically-acceptable mutual prodrug compounds of the present invention to a subject.

In embodiments of the methods of treating cancer, the cancer includes epithelial tumors, melanoma, leukemia, such as acute promyelocytic leukemia, lymphoma, osteogenic sarcoma, colon cancer, pancreatic cancer, breast cancer, prostate cancer, ovarian cancer, and lung cancer.

In embodiments of the methods of treating cancer, the one or more pharmaceutically-acceptable mutual prodrug compounds may be administered in combination with other active agents. Combination therapy includes combining the method of treating cancer as described in the invention and one or more cancer therapeutic methods. Cancer therapeutic methods include surgical therapy, radiation therapy, administering an anticancer agent (including, for example, antineoplastics (including, for example, novantrone, bicalutamide, esterified estrogens, goserelin, histrelin, leuprolide, nilandron, triptorelin pamoate, docetaxel, taxotere, carboplatin, and cisplatin) or combinations thereof, and angiogenesis inhibitors), immunotherapy, antineoplastons, investigational drugs, vaccines, less conventional therapies (sometimes referred to as novel or innovative therapies, which include, for example, chemoembolization, hormone therapy, local hyperthermia, photodynamic therapy, radiofrequency ablation, stem cell transplantation, and gene therapy), prophylactic therapy (including, for example, prophylactic mastectomy or prostatectomy), and alternative and complementary therapies (including, for example, dietary supplements, megadose vitamins, herbal preparations, special teas, physical therapy, acupuncture, massage therapy, magnet therapy, spiritual healing, meditation, pain management therapy, and naturopathic therapy (including, for example, botanical medicine, homeopathy, Chinese medicine, and hydrotherapy)).

The present invention includes methods of treating a dermatologic condition using one or more of the mutual prodrug compounds of the present invention. In particular, the present invention includes methods of treating a dermatologic condition in a subject in need of treatment, comprising administering one or more of the mutual prodrug compounds of the present invention to a subject. In a preferred embodiment, the present invention includes methods of treating a dermatologic condition in a subject in need of treatment, comprising administering one or more of the pharmaceutically-acceptable mutual prodrug compounds of the present invention to a subject.

In embodiments of the methods of treating a dermatologic condition, the dermatologic condition includes acne, psoriasis, wrinkling, and photoaged skin.

In embodiments of the methods of treating a dermatologic condition, the one or more pharmaceutically-acceptable mutual prodrug compounds may be administered in combination with other active agents.

The present invention also includes the use of one or more of the pharmaceutically acceptable mutual prodrug compounds of the present invention as a medical treatment of cancer.

The present invention further includes the use of one or more of the pharmaceutically-acceptable mutual prodrug compounds of the present invention in the manufacture of a medicament for treatment of cancer or a dermatologic condition.

The present invention also includes a kit comprising one or more of the mutual prodrug compounds of the present invention and instructions for its use. Similarly, the present invention includes a kit comprising one or more of the pharmaceutically acceptable mutual prodrug compositions of the present invention and instructions for its use.

One goal of the present invention is to develop mutual prodrugs that might exert stronger antiproliferative activity in cancer cells than co-treatments of the two agents which comprise the mutual prodrugs, or than each of the two agents alone.

### BRIEF DESCRIPTION OF THE DRAWINGS

FIG. 1A: G1₅₀ determinant for CI-994.

FIG. 1B: GI₅₀ determinant for VNLG/34-B.

FIG. 1C: GI₅₀ determinant for mutual prodrug VNLG/60.

FIG. 2A The HPLC chromatogram shown demonstrating the absence of mutual prodrug in plasma after one hour and presence of ATRA

FIG. 2B The HPLC chromatogram shown demonstrating the absence of mutual prodrug in plasma after one hour and presence of MS-275.

FIG. 3: Mechanism of drug release from Prodrug with 1,6 elimination system.

FIG. 4: Concentration-dependent curve showing the antiproliferative effect of mutual prodrug VNGL/122 (18) on human prostate cancer PC-3 cells. Data are means (±SEM) of at least three independent experiments. The experiments with the other compounds gave plots that were similar to that shown above.

FIG. 5: Effect of ATRA and MS-275 administered alone or in combination and of corresponding mutual prodrug VNLG/114 on MDA-MB-231 cell growth. * indicates a significant increase from control and MS-275, ATRA or MS-275+ ATRA treatments (P<0.01).

FIG. 6: Effect of ATRA and CI-994 administered alone or in combination and of corresponding mutual prodrug VNLG/122 on PC3 cell growth. Data are means (±SEM) of at least three independent experiments.

FIG. 7: Growth inhibitory effect of 3 and VNLG/124 (19) in MDA-MB-231 breast cancer cell line. Data are means (±SEM) of at least three independent experiments.

FIG. 8: Effect of ATRA and butyric acid administered alone or in combination and of VNLG/124 (19) on PC3 cell growth. ** indicates a significant increase from control and BA, ATRA or BA+ATRA treatments (P<0.001).

FIG. 9: Growth inhibitory effect of ATRA and sodium butyrate in combination and of corresponding VNLG/124 (19) in PC-3 cell line. Data are means (±SEM) of at least three independent experiments.

FIG. 10: Effect of ATRA and butyric acid administered alone or in combination and of mutual prodrug 19 on MDA-MB-231 cell growth. *** indicates a significant increase from ATRA (5 nM)+BA (5 n) treatment (P<0.0001). ### indicates a significant increase from ATRA (10 nM)+BA (10 nM) treatment (P<0.0001).

FIG. 11: Chemical structures of compounds 1-3, butyric acid (BA), and all-trans retinoic acid (ATRA)

### DETAILED DESCRIPTION OF THE INVENTION

### HDACIs

The HDACIs in the compounds of the present invention may be any known HDACI in the art. Non-limiting examples of HDACIs to be linked in the compounds of the present invention include N-hydroxy-N¹-phenyloctanediamide also, called suberoylanilide hydroxamic acid (SAHA):

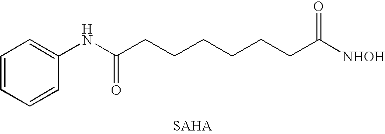

### N-(2′-aminophenyl)-4-acetylaminobenzamide (also know as CI-994):

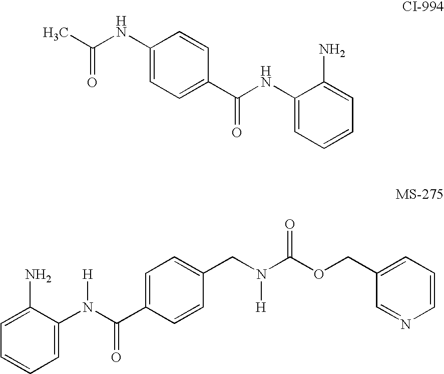

and N-(2-aminophenyl)-4-[N-(pyridin-3-yl-methoxycarbonyl)aminomethyl]benzamide.

### Retinoids

All-trans retinoic acid (ATRA) is a well-known and characterized compound. Its catabolic pathway involves ATRA 4-hydroxylase. The iron oxene species (Fe^{v}═O) of ATRA 4-hydroxylase is responsible for molecular oxygen activation and thus, the breakdown of ATRA. The Fe^{v}═O group of ATRA 4-hydroxylase has access to the C-4 of ATRA in that C-4 is within bonding distance of the activated oxygen. Substitution of suitable groups at the C-4 of ATRA will generate ATRA analogs which both react with the retinoid-binding site of the enzyme and interact with the heme iron and/or the protein residue with high specificity. Substitutions of suitable groups can increase the inhibitory affects of the new compounds with Kᵢ values in the nanomolar range.

The retinoids to be linked in the compounds of the present invention may be any known retinoids in the art. Non-limiting examples of the retinoids to be linked in the compounds of the present invention include all-trans retinoic acid (ATRA):

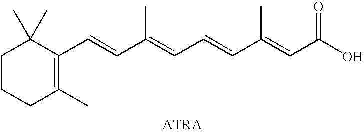

### 13-cis retinoic acid (13-CRA)

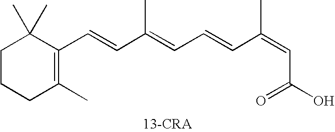

as well as derivatives thereof as described below

### RAMBAs

The RAMBAs to be linked in the compounds of the present invention may be any RAMBAs known in the art, including both C-4 substituted ATRA analogs and C-4 substituted 13-CRA analogs. Exemplary RAMBAs to be linked in the compounds of the present invention include compounds of formula (I):

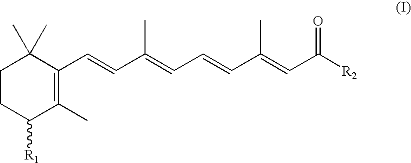

wherein

R₁ is an azole group, an allylic azole group, a sulfur-containing group, an oxygen-containing group, a nitrogen-containing group, a pyridyl group, an ethinyl group, a cyclopropyl-amine group, an ester group, an amino group, an azido group, or a cyano group, or R₁ forms, together with the C-4 carbon atom, an oxime, an oxirane or aziridine group;

R₂ is a hydroxyl group, an aminophenol group, an ester group, an azole group or —OR₃, wherein R₃ is selected from the group consisting of an alkyl, an aryl and a heterocyclic group; and

wherein, independently, any of the unsaturations may be cis or trans.

Preferred RAMBA compounds of formula (I) include those set forth in Table 1.

TABLE 1

| Compound | R₁ | R₂ |
|---|---|---|
| VN/12-1^{t} | 1H-imidazole | —OCH₃ |
| VN/13-1^{t} | 1H-1,2,4-triazole | —OCH₃ |
| VN/13-2^{t} | 2H-1,2,4-triazole | —OCH₃ |
| VN/14-1^{t} | 1H-imidazole | —OH |
| VN/16-1^{t} | 1H-1,2,4-triazole | —OH |
| VN/17-1^{t} | 2H-1,2,4-triazole | —OH |
| VN/50A-1^{t} | 1H-imidazole | 1H-imidazole |
| VN/51A-1^{t} | Keto oxime | —OCH₃ |
| VN/66-1^{t} | 1H-imidazole | —NHC₆H₄OH |
| VN/65-4* | 1H-imidazole | —OCH₃ |
| VN/67-1* | 1H-imidazole | —OH |
| VN/68-1* | 1H-imidazole | 1H-imidazole |
| VN/69-1* | 1H-imidazole | —NHC₆H₄OH |

^{t}C-4 substituted ATRA analogs.
*C-4 substituted 13-CRA analogs.

Other, non-limiting examples of RAMBAs include those described in U.S. Pat. No. 7,265,143 issued Sep. 4, 2007, which is hereby incorporated by reference in its entirety.

In embodiments where R₁ is a sulfur-containing group, preferably R₁ is thiirane, thiol or a alkylthiol derivative, more preferably R₁ is a C₁ to C₁₀ alkyl thiol.

In embodiments where R₁ is an oxygen-containing group, preferably is R₁—OR₄, where R₄ is hydrogen or an alkyl group, cyclopropylether or an oxygen containing group that forms, together with the 4-position carbon, an oxirane group. In preferred embodiments, the alkyl is a 1-10 carbon alkyl, more preferably R₁ is methyl or ethyl.

In embodiments where R₁ is a nitrogen-containing group, preferably R₁ is —NR₅R₆, where R₅ and R₆ are independently selected from the group consisting of hydrogen and an alkyl group, or R₅ and R₆ together form a 3-6 membered ring that may include a heteroatom selected from N, S, and O. In an equally preferred embodiment, the alkyl group is a 1-10 carbon alkyl, more preferably methyl or ethyl. In a further preferred embodiment, the ring formed by R₅ and R₆ is an imidazolyl ring or a triazole ring.

In embodiments where R₁ is an azole group, preferably R₁ is an imidazole or a triazole, including those attached through a nitrogen ring atom, more preferably R₁ is 1H-imidazole-1-yl, 1H-1,2,4-triazol-1-yl or 4H-1,2,4-triazol-1-yl.

In embodiments where R₁ is an allylic azole group, preferably R₁ is methyleneazolyl.

In embodiments where R₁ is an ester, the ester may include a substituent group that contains an ester moiety, including a substituent group attached via an ester moiety.

In embodiments where R₂ is —OR₃, preferably R₃ is hydroxyl or a straight or branched chain alkyl selected from the group consisting of methyl, ethyl, propyl and butyl.

In exemplary embodiments of formula I, the double bond between C-13 and C-14 is cis or trans.

The term “alkyl” includes substituted and unsubstituted alkyl groups, branched and straight chain and cyclo alkyl groups, such as cyclopropyl.

The term “aryl” includes a phenyl or naphthyl ring.

The term “heterocyclic group” includes an unsubstituted or substituted stable 3- to 7-membered monocyclic or 7- to 10-membered bicyclic heterocyclic ring and which consists of carbon atoms and from one to three heteroatoms selected from the group consisting of nitrogen, oxygen and sulfur, and wherein the nitrogen and sulfur heteroatoms may optionally be oxidized, and the nitrogen heteroatom may optionally be quaternized and including a bicyclic group in which any of the above-defined heterocyclic rings is fused to a benzene ring. The heterocyclic ring may be attached at any heteroatom or carbon atom which affords a stable structure. The heterocyclic group may be saturated or unsaturated.

Examples of heterocyclic groups include piperidinyl, piperazinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazolyl, benzopyranyl, benzothiazolyl, benzoazolyl, faryl, tetrahydrofuryl, tetrahydropyranyl, thienyl, benzothienyl, thiamorpholinyl, thiamorpholinylsulfoxide, thiamorpholinylsulfone, oxadiazolyl, triazolyl, tetrahydroquinolinyl, and tetrahydroisoquinolinyl.

For ATRA analogs with C-4 substitutions with azole, sulfur, oxygen, or nitrogen, following binding at the active-site of the 4-hydroxylase enzyme, the lone pair of electrons coordinate to the prosthetic heme iron causing inhibition of the enzyme. Blockage of ATRA 4-hydroxylase activity increases the amount of ATRA.

The precursors for VN/65-4 and VN/66-1 are those of 13-cis-retinoic acid (13-CRA) and fenretinide, respectively. These compounds have long elimination half-lives in most animal species, and thus are believed to have improved pharmacokinetic (PK) parameters. VN/65-4 and VN/66-1 have excellent ATRA 4-hydroxylase inhibitory activity and favorable pharmacokinetic properties.

In preferred embodiments, the RAMBA compounds of formula (I) inhibit the activity of ATRA 4-hydroxylase.

### Mutual Prodrugs—Linked

In preferred aspects, the present invention includes mutual prodrugs where one or more HDACIs is chemically linked to one or more retinoids, one or more HDACIs is chemically linked to one or more RAMBAs, or one or more HDACIs is chemically linked to both one or more retinoids and one or more RAMBAs.

The clinical development of retinoids in the treatment of cancer, such as epithelial tumors, has been hampered by the development of resistance. Loss of retinoic acid sensitivity has been associated with lack of RARβ2 expression. In the presence of retinoids, HDACIs induce acetylation in RARβ2 hypermethylated promoters, leading to the re-expression of RARβ2 in RARβ2-negative, retinoid-resistant tumor cells. While studies have indicated that the co-administration of retinoids and HDACIs can be effective inhibitors of cellular proliferation, the individual agents can be transported to the site of action with different efficiencies. Mutual prodrugs comprising chemically linked components can be transported to the site of action with the same efficiency and release both drugs in vivo.

The mutual prodrugs may be chemically synthesized to various starting materials to produce the mutual prodrugs of the present invention. While particular methods of synthesis are provided in the Example, the skilled artisan will understand that a number of different methods may be used to chemically synthesize the mutual prodrugs of the present invention.

Preferably, the mutual prodrugs should not release any toxic agent other than the parent drug or drugs. To achieve this goal, glycine acyloxyalkyl carbamate linkers (AC-linkers) and benzyl ester linkers (elimination linkers) may be used. The 1,6 elimination or the 1,4 elimination concept may be used for preparing the mutual prodrugs of the present invention. The 1,4 or 1,6 elimination concept has been used for preparing prodrugs of many anticancer compounds for tumor targeted drug delivery. In order to exploit the synergistic effect of HDACIs and retinoids and/or RAMBAs more effectively, mutual prodrugs of the present invention may be prepared using the 1,6 concept.

Generally, 1,6 elimination of HX (where X is a good leaving group like halide, functionalized oxygen derivatives such as carboxylates, or a carbamic acid anion) from benzyl compounds bearing strong electron-releasing para-hydroxy or para-amino substituents is a fast reaction that occurs under mildly basic conditions. Concomitantly, quinone methides and quinonimine methides are produced. Further quinone methides are converted into p-hydroxy benzylalcohol by addition of water.

Non-limiting embodiments of a mutual prodrug compounds comprising (i) one or more HDACIs and (ii) one or more retinoic acids that are chemically linked are:

MS-275 and ATRA.

MS-275 and VN/66-1.

MS-275 and VN/14-1.

MS-275 and VN/12-1.

MS-275, 1,6 elimination linker and ATRA.

MS-275, 1,6 elimination linker and VN/66-1.

MS-275, 1,6 elimination linker and VN/14-1.

MS-275, 1,6 elimination linker and VN/12-1.

MS-275, acyloxymethylcarbamate linker and ATRA.

MS-275, acyloxymethylcarbamate linker and VN/66-1.

MS-275, acyloxymethylcarbamate linker and VN/14-1.

MS-275, acyloxymethylcarbamate linker and VN/12-1.

CI-994 and ATRA.

CI-994 and VN/66-1.

CI-994 and VN/14-1.

CI-994 and VN/12-1.

CI-994, 1,6 elimination linker and ATRA.

CI-994, 1,6 elimination linker and VN/66-1.

CI-994, 1,6 elimination linker and VN/14-1.

CI-994, 1,6 elimination linker and VN/12-1.

CI-994, acyloxymethylcarbamate linker and ATRA.

CI-994, acyloxymethylcarbamate linker and VN/66-1.

CI-994, acyloxymethylcarbamate linker and VN/14-1.

CI-994, acyloxymethylcarbamate linker and VN/12-1.

SAHA and ATRA.

SAHA and VN/66-1.

SAHA and VN/14-1.

SAHA and VN/12-1.

SAHA, 1,6 elimination linker and ATRA.

SAHA, 1,6 elimination linker and VN/66-1.

SAHA, 1,6 elimination linker and VN/14-1.

SAHA, 1,6 elimination linker and VN/12-1.

SAHA, acyloxymethylcarbamate linker and ATRA.

SAHA, acyloxymethylcarbamate linker and VN/66-1.

SAHA, acyloxymethylcarbamate linker and VN/14-1.

SAHA, acyloxymethylcarbamate linker and VN/12-1.

Non-limiting examples of a mutual prodrug compounds comprising (i) one or more HDACIs and (ii) one or more retinoic acids that are chemically linked are:

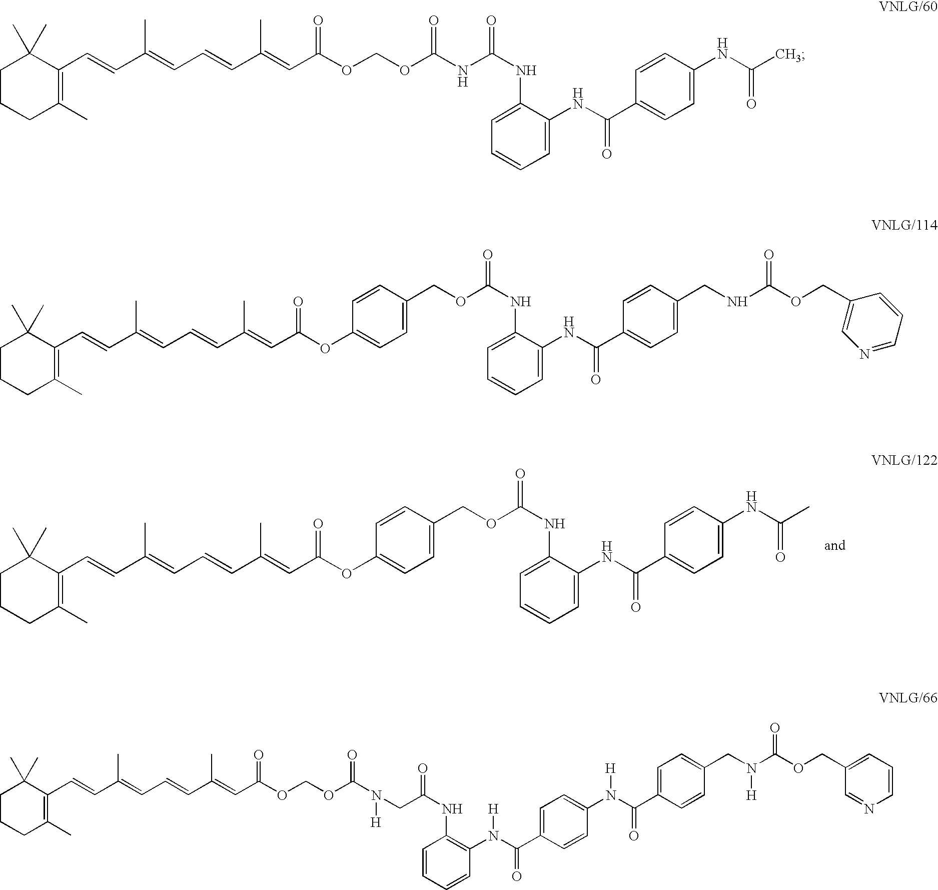

### Pharmaceutically Acceptable Compositions

In related embodiments of the present invention, the mutual prodrug compounds may be formulated in pharmaceutically acceptable mutual prodrug compositions, comprising one or more of the mutual prodrug compositions of the present invention and a pharmaceutically acceptable carrier or an excipient.

The mutual prodrugs can be mixed with a pharmaceutically acceptable carrier or an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier, or medium for the mutual prodrug. Thus, the pharmaceutically acceptable mutual prodrug compositions can be in the form of tablets, pills, powers, lozenges, sachets, cachets, elixirs, suspensions, emulsions, solutions, syrups, soft and hard gelatin capsules, and other orally ingestible formulations.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, magnesium carbonate, water, ethanol, propylene glycol, syrup, and methyl cellulose. The formulations can additionally include lubricating agents such as talc, magnesium stearate, and mineral oil; wetting agents; emulsifying and suspending agents; preserving agents such as methyl- and propyl-hydroxybenzoates, sweetening agents; and flavoring agents. The compositions of the present invention can also be formulated so as to provide quick, sustained or delayed release of the novel compound after administration to the patient by employing procedures known in the art.

The term “pharmaceutically acceptable carrier” refers to those components in the particular dosage form employed which are considered inert and are typically employed in the pharmaceutical arts to formulate a dosage form containing a particular active compound. This may include without limitation solids, liquids and gases, used to formulate the particular pharmaceutical product. Examples of carriers include diluents, flavoring agents, solubilizers, suspending agents, binders or tablet disintegrating agents, encapsulating materials, penetration enhancers, solvents, emollients, thickeners, dispersants, sustained release forms, such as matrices, transdermal delivery components, buffers, stabilizers, and the like. Each of these terms is understood by those of ordinary skill.

The pharmaceutically acceptable mutual prodrug compositions may be formulated in the absence or presence of carriers or excipients that provide a sustained released formulation.

The pharmaceutically acceptable mutual prodrug compositions may be prepared according to methods well known in the art. It is contemplated that administration of such compositions may be by the oral, injectable and/or parenteral routes depending upon the needs of the artisan. The pharmaceutically acceptable mutual prodrug compositions can also be administered by nasal or oral inhalation, oral ingestion, injection (intramuscular, intravenous, and intraperitoneal), transdermally, or other forms of administration.

Aerosol formulations for use in this invention typically include propellants, such as a fluorinated alkane, surfactants and co-solvents and may be filled into aluminum or other conventional aerosol containers which are then closed by a suitable metering valve and pressurized with propellant, producing a metered dose inhaler. Aerosol preparations are typically suitable for nasal or oral inhalation, and may be in powder or solution form, in combination with a compressed gas, typically compressed air. Additionally, aerosols may be useful topically.

Topical preparations useful herein include creams, ointments, solutions, suspensions and the like. These may be formulated to enable one to apply the appropriate dosage topically to the affected area once daily, up to 3-4 times daily as appropriate. Topical sprays may be included herein as well.

Depending upon the particular mutual prodrug selected, transdermal delivery may be an option, providing a relatively steady state delivery of the medication which is preferred in some circumstances. Transdermal delivery typically involves the use of a compound in solution, with an alcoholic vehicle, optionally a penetration enhancer, such as a surfactant and other optional ingredients. Matrix and reservoir type transdermal delivery systems are examples of suitable transdermal systems. Transdermal delivery differs from conventional topical treatment in that the dosage form delivers a systemic dose of medication to the patient.

### Methods of Treatment

The present invention includes methods of inhibiting the activity of ATRA 4-hydroxylase using one or more of the mutual prodrug compounds of the present invention. In particular, the present invention includes methods of inhibiting the activity of ATRA 4-hydroxylase in a subject comprising administering one or more of the mutual prodrug compounds of the present invention to a subject. In a preferred embodiment, the present invention includes methods of inhibiting the activity of ATRA 4-hydroxylase in a subject comprising administering one or more of the pharmaceutically-acceptable mutual prodrug compounds of the present invention to a subject.

The present invention includes methods of treating cancer using one or more of the mutual prodrug compounds of the present invention. In particular, the present invention includes methods of treating cancer in a subject in need of treatment, comprising administering one or more of the mutual prodrug compounds of the present invention to a subject. In a preferred embodiment, the present invention includes methods of treating cancer in a subject in need of treatment, comprising administering one or more of the pharmaceutically-acceptable mutual prodrug compounds of the present invention to a subject.

In embodiments of the methods of treating cancer, the cancer includes epithelial tumors, melanoma, leukemia, such as acute promyelocytic leukemia, lymphoma, osteogenic sarcoma, colon cancer, pancreatic cancer, breast cancer, prostate cancer, ovarian cancer, and lung cancer.

In embodiments of the methods of treating cancer, the one or more pharmaceutically-acceptable mutual prodrug compounds may be administered in combination with other active agents.

The present invention includes methods of treating a dermatologic condition using one or more of the mutual prodrug compounds of the present invention. In particular, the present invention includes methods of treating a dermatologic condition in a subject in need of treatment, comprising administering one or more of the mutual prodrug compounds of the present invention to a subject. In a preferred embodiment, the present invention includes methods of treating a dermatologic condition in a subject in need of treatment, comprising administering one or more of the pharmaceutically-acceptable mutual prodrug compounds of the present invention to the subject.

In embodiments of the methods of treating a dermatologic condition, the dermatologic condition includes acne, psoriasis, wrinkling, and photoaged skin.

In embodiments of the methods of treating a dermatologic condition, the one or more pharmaceutically-acceptable mutual prodrug compounds may be administered in combination with other active agents.

Generally, the amount of the mutual prodrug compound used in the methods of treatment is that amount which effectively achieves the desired therapeutic result in a subject, whether the mutual prodrug compound is administered as a pharmaceutically acceptable composition, or in the absence of a carrier or diluent. Naturally, the dosages of the various mutual prodrugs will vary somewhat depending upon the components of the prodrugs, the rate of in vivo hydrolysis, etc. Those skilled in the art can determine the optimal dosing of the novel compound selected based on clinical experience and the treatment indication.

Preferably, the amount of the mutual prodrug in the compounds administered to a subject, whether the mutual prodrug compound is administered as a pharmaceutically acceptable composition, or in the absence of a carrier or diluent, is about 0.1 to about 100 mg/kg of body weight, more preferably, about 5 to about 40 mg/kg. Other preferred dosages include about 0.1 to about 10 mg/kg of body weight, about 1 to about 100 mg/kg of body weight, about 1 to about 60 mg/kg of body weight, about 1 to about 10 mg/kg of body weight, about 10 to about 100 mg/kg of body weight, about 10 to about 60 mg/kg of body weight, about 20 to about 60 mg/kg of body weight and about 30 to about 50 mg/kg of body weight.

The mutual prodrugs can also be converted into a pharmaceutically acceptable salt or pharmaceutically acceptable solvate or other physical forms (e.g., polymorphs by way of example only and not limitation) via known in the art field methods.

The subjects that may be treated using the compounds methods of the present invention include mammals, such as humans.

As used herein, the terms “inhibit”, “inhibiting” and “inhibition” include complete inhibition, as well as a partial inhibition, such as 50%, 60%, 70%, 80%, 90% or 95% inhibition.

As used herein, the terms “treat”, “treating” and “treatment” include the achievement of a complete absence of symptoms, as well as a decrease in symptoms, of a disease or condition for which the compounds of the present invention are being administered. These terms also refer to a complete cure, as well as a decrease in the severity, of a disease or condition for which the compounds of the present invention are being administered. These terms also refer to a decrease in the duration of a disease or condition for which the compounds of the present invention are being administered. Further, these terms apply to subjects that have a disease or condition for which the compounds of the present invention are being administered, as well as those subjects that are at risk for developing or contracting a disease or condition for which the compounds of the present invention are being administered.

Other active agents that may be administered in conjunction with the compounds of the present invention include those compounds known in the art to be useful in the treatment of cancer and dermatologic diseases and conditions.

The present invention also includes the use of one or more of the pharmaceutically acceptable mutual prodrug compounds of the present invention as a medical treatment of cancer.

The present invention further includes the use of one or more of the pharmaceutically-acceptable mutual prodrug compounds of the present invention in the manufacture of a medicament for treatment of cancer or a dermatologic condition.

The present invention also includes a kit comprising one or more of the mutual prodrug compounds of the present invention and instructions for its use. Similarly, the present invention includes a kit comprising one or more of the pharmaceutically acceptable mutual prodrug compounds of the present invention and instructions for its use.

### Examples

Inhibition of Prostate Cancer Proliferation In-Vitro

The novel C-4 substituted ATRA analogs inhibit proliferation of prostate cancer in-vitro. Experiments were conducted on two prostate cancer cell lines, LNCaP cells and PC-3 cells. LNCaP cells are androgen-dependent cell cultures. PC-3 cells are androgen independent cell culture. LNCaP cells harbor both wild-type p53 and RB tumor-suppressor genes while PC-3 cells only express the wild-type RB gene and are null of p53 protein as a result of mutation. Thus, these two cell lines were used as representatives of hormone-dependent and independent human prostate cancer.

Methods

LNCaP cells were transferred into ATRA-free medium 3 days prior to start of experiments. Medium consisted of phenol red-free IMEM supplemented with 5% FBS and 1% P/S. Cell were then plated into 24-well culture plates (15000 cells per well) in 1 mL of same medium. After a 24-hour attachment period, the vehicle (ethanol) or ATRA (10⁻⁵ M) alone or ATRA in combination with a novel compound at a range of concentrations were added to triplicate wells. Medium/treatments were changed every 3 days. After 9 days of treatment, cells were removed from the wells with typsin/EDTA and counted in a Coulter counter. Using well-known methods (Wouters W, Van Dun J, Dillen A, Coene M.-C, Cools W and De Coster R, Effects of liarozole, a new antitumoral compound an retinoic acid-induced inhibition of cell growth and on retinoic acid metabolism in MCF-7 breast cancer cells, Cancer Res 52: 2841-2846, 1992), the inhibitory effect of the novel compounds on LNCaP cells grown with ATRA was determined.

This method also was repeated using PC-3 cells.

### Cell Culture

PC-3 (androgen receptor negative, AR−ve) cells were obtained from American Type Culture Collection (ATCC, Rockville, Md., USA). Cells were maintained in RPMI 1640 medium (Gibco, Invitrogen, Carlsbad, Calif., USA) supplemented with 10% fetal bovine serum (Atlanta Biologicals, Lawrenceville, Ga., USA) and 1% penicillin/streptomycin. Cells were grown as a monolayer in T75 tissue culture flasks in a humidified incubator (5% CO₂, 95% air) at 37° C.

Other cell lines used in this study include, MDA-MB-231 (estrogen receptor negative, ER−ve), MCF-7 (ER+ve) were also purchased from ATCC and were cultured as previously described.^{44, 45} LTLC and LTLT-Ca were kindly provided by Dr. Angela Brodie, University of Maryland, Baltimore, and details of their phenotypes and culturing conditions are as previously reported.^{46, 47} MCF-7_{TAM} and MCF-7_{HoxB-7} were provided by Dr. Sara Sukumar of Johns Hopkins University, Baltimore, and details of their phenotypes and culturing conditions are as previously reported.⁴⁸ Except for MCF-7 breast cancer cell all other breast cancer cells used in this study are insensitive to endocrine therapeutic agents and to most anti-cancer agents.

### Cell Growth Inhibition (MTT Colorimetric Assay)

PC-3 cells were seeded in 24 well plates (Corning Costar) at a density of 2×10⁴ cells per well per 1 mL of medium. Cells were allowed to adhere to the plate for 24 hours and then treated with different concentrations of ATRA, HDIs or mutual prodrugs dissolved in 10% DMSO, 90% ethanol. Cells were treated for five days with renewal of prodrug and media on day 3. On the fifth day, medium was renewed and 100 μL of MTT (3-(4,5-dimethylthiazol-2-yl)-2,5-diphenyl-2H-tetrazolium bromide from Sigma) solution (0.5 mg MTT/mL of media) was added to the medium such that the ratio of MTT:medium was 1:10. The cells were incubated with MTT for 2 hours. The medium was then aspirated and 500 μL of DMSO was added to solubilize the violet MTT-formazan product. The absorbance at 560 nm was measured by spectrophotometry (Victor 1420 multilabel counted, Wallac). For each concentration of agent or mutual prodrugs there were triplicate wells in each independent experiment. GI₅₀ values were calculated by nonlinear regression analysis using GraphPad Prism software.

VNLG/60

The mutual prodrug VNLG/60 is a combination of the HDACI CI-994 (N-(2′-aminophenyl)-4-acetylaminobenzamide) and the retinoic acid VNLG/34-B (derived from ATRA). VNLG/60 was produced in the manner shown in Schemes 1 and 2.

### 1. Chemistry

All the reagents were purchased from Aldrich. Precoated silica gel GF plates from Analtech were used for TLC and observed under UV. Flash column chromatography was performed on silica gel 60. Infrared spectra were recorded on Perkin-Elmer 1600 IR spectrometer using Nujol paste. 1H NMR spectra were performed in CDCl3 and DMSOd6 at 500 MHz with Me4Si as an internal standard using a Varian Inova 500 MHz spectrometer. Mass spectra were recorded in the positive ion mode with an ESI-probe on Quattro micro triple quadrupole mass spectrometer (Micromass-Waters). CI-994 was produced CI-994 at high purity (98% by HPLC) and yield (80%).

### 2. Biology

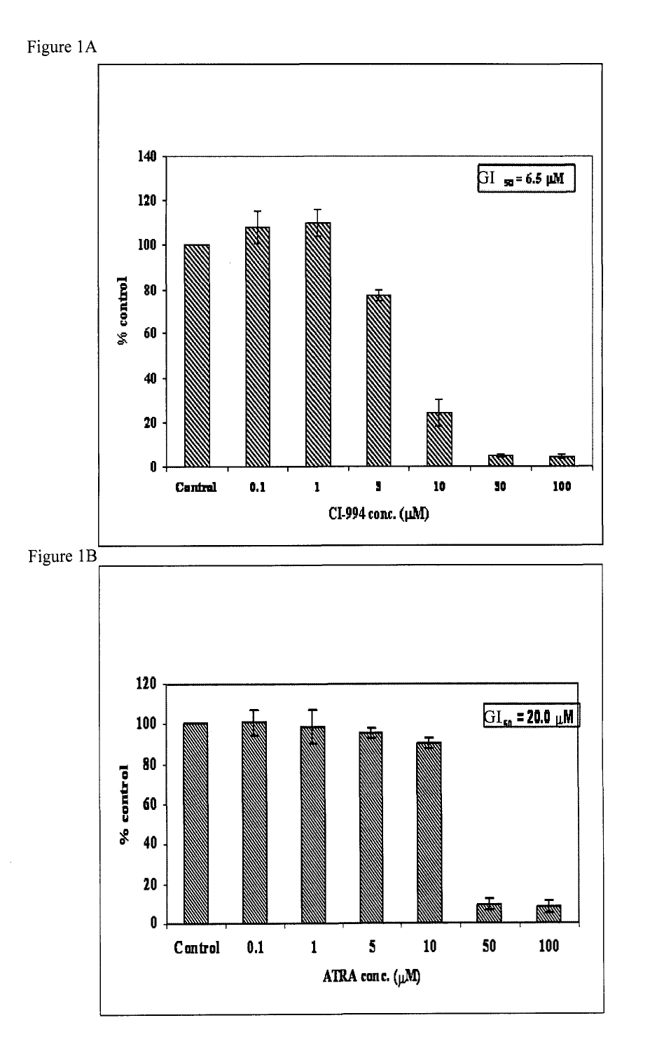

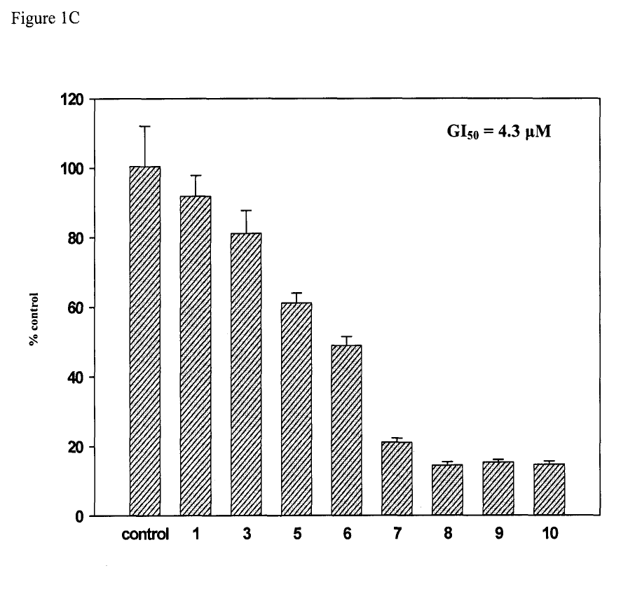

When applied to the hormone independent breast cancer cell line MDA-MB-231, the mutual prodrug VNLG/60 showed a synergistic growth inhibitory effect, with an GI₅₀ of 4.3 uM, by MTT Colorimetric Cell Viability Assay (FIG. 1C). The MTT (3-[4,5-dimethylthiazol-2-yl]-2,5-diphenyltetrazolium bromide) assay was used to measure the cell viability. MDA-MB-231 cells (1.0×10³ cells/ml) were seeded into 96 well plates. Cells were treated with the compound on day 2 at different concentrations. On the 5th day, the medium was removed and 1 mL of a 0.5 mg/ml MTT solution was added to each well. The plates were incubated at 37° C. for 2 hours and 200 μL of DMSO was added. The yellow tetrazolium salt (MTT) is reduced to a purple formazan product by the mitochondrial dehydrogenase from living cells. The product is then detected by spectrophotometry at 560 nm. The IC₅₀ of CI-994 is shown in FIG. 1A, and IC₅₀ of VNLG/34-B is shown in FIG. 1B.

### VNLG/114 and VNLG/122

### 1. Materials and Methods

All the reagents were purchased from Aldrich. Pre-coated silica gel GF plates from Analtech were used for TLC and observed under UV. Flash column chromatography was performed on silica gel 60. Infrared spectra were recorded on Perkin-Elmer 1600 IR spectrometer using Nujol paste. 1H NMR spectra were performed in CDCl3 and DMSOd6 at 500 MHz with Me4Si as an internal standard using a Varian Inova 500 MHz spectrometer. Mass spectra were recorded in the positive ion mode with an ESI-probe on Quattro micro triple quadrupole mass spectrometer (Micromass-Waters). HPLC system used in this study consisted of a solvent delivery system, controller (Milford, Mass.) coupled to a 717plus auto-sampler, and 996 photodiode array detector (all from Waters). Chromatographic analysis was achieved by a reverse-phase HPLC method on a Waters Novapak C18 column (3.9 mm×150 mm).

### 2. Chemistry

Synthesis of mutual prodrugs VNLG/114 and VNLG/122 is outlined in Scheme 5. First step is the preparation of p-hydroxy benzaldehyde ester of ATRA (compound 15) by coupling p-hydroxy benzaldehyde with ATRA using DCC/DMAP. Reduction of aldehyde 15 with sodium borohydride gave benzyl alcohol 16. Benzyl alcohol 16 was reacted with triphosgene in toluene using sodium carbonate as base to give a chloroformate derivative. The chloroformate derivative was further condensed with MS-275 to produce the desired mutual prodrug, VNLG/114. Similar reaction with CI-994 yielded VNLG/122.

### 3. Kinetics of Hydrolysis in Plasma

Plasma was obtained by centrifugation of blood samples (obtained from mice) containing 0.25% heparin. 0.8 ml of plasma was mixed with 0.2 ml of phosphate buffer (pH 7.2). Incubations were performed at 37° C.+/−0.5° C. using shaking water bath. The reaction was initiated by adding 25 μl of stock solution of mutual prodrugs (1 mg/ml) to preheated plasma and aliquots were taken at different time interval and extracted using solid phase extraction with C18 Bond Elut columns. HPLC analysis of the extracted samples were performed using a multi-linear gradient solvent system, (i) 20 mM aq. CH₃COONH₄ buffer/CH₃OH(50:50), (100% to 0%) and (ii) CH₃OH(100%), (0% to 100%) at a flow rate of 0.8 ml/min. Retention time for ATRA, MS-275 and mutual prodrug VNLG/114 were 21.852, 3.087 and 25.634 min. respectively.

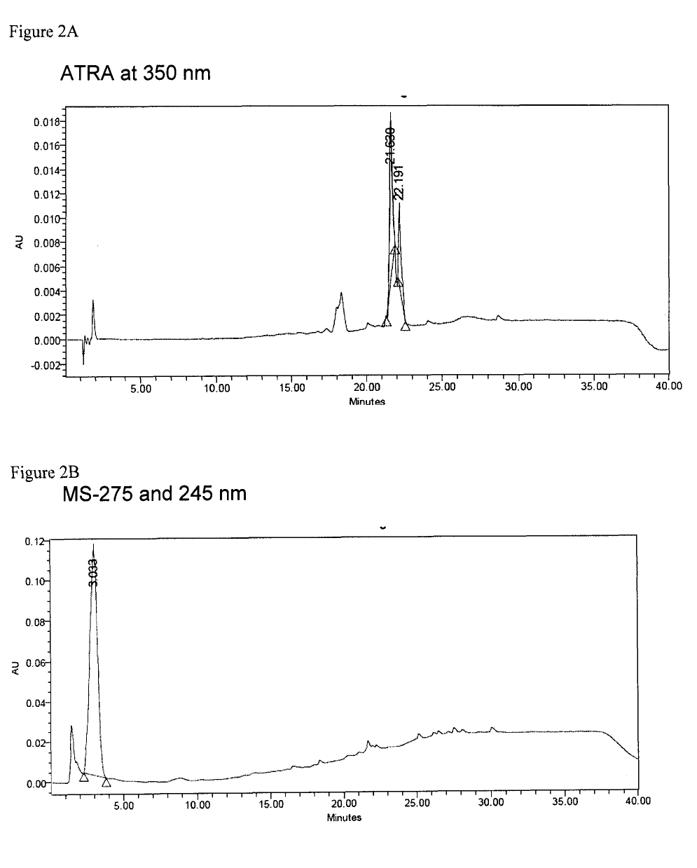

The HPLC chromatogram shown in FIGS. 2A and 2B demonstrates the absence of mutual prodrug in plasma after one hour and presence of ATRA (FIG. 2A) and MS-275 (FIG. 2B). The mutual prodrugs were cleaved after one hour. Hence, these mutual prodrugs are bio-reversible and will release individual drugs in vivo. All the mutual prodrugs were stable in phosphate buffer for 36 hours.

### 4. MTT Colorimetric Cell Viability Assay

The MTT (3-[4,5-dimethylthiazol-2-yl]-2,5-diphenyltetrazolium bromide) assay was used to measure the cell viability. PC-3 cells or MDA-MB-231 cells (1.0×10³ cells/ml) were seeded into 96 well plates. Cells were treated with the compounds on day 2 at different concentrations. On the 5th day, the medium was removed and 1 mL of a 0.5 mg/ml MTT solution was added to each well. The plates were incubated at 37° C. for 2 hours and 200 μL of DMSO was added. The yellow tetrazolium salt (MTT) is reduced to a purple formazan product by the mitochondrial dehydrogenase from living cells. The product is then detected by spectrophotometry at 560 nm.

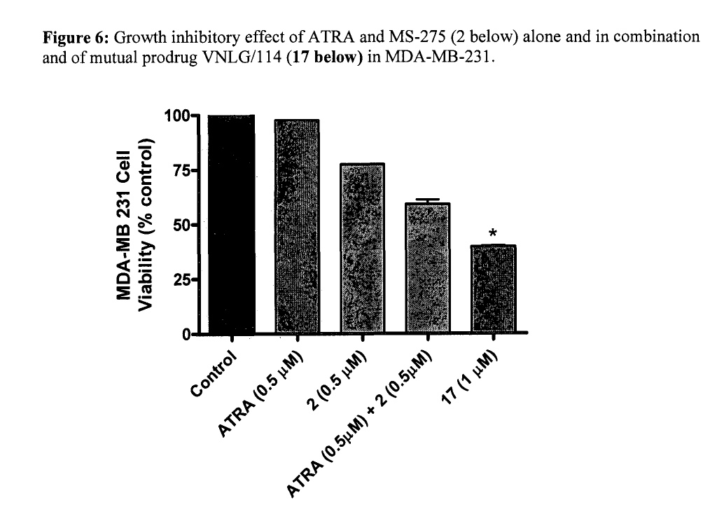

VNLG/114 showed synergistic growth inhibitory effect in MDA-MB-231 breast cancer cells (p<0.0001) (FIG. 6). Results of GI₅₀ determinations are shown in Table 2. GI₅₀=concentration of agent that causes 50% cell growth inhibition.

TABLE 2
GI₅₀ values obtained from dose-response curves in PC-3 and
MDA-MB-231 cell lines:

| | GI₅₀ Values (μM)^{a} | |
|---|---|---|
| | PC-3 prostate | MDA-MB-231 |
| | cancer | breast cancer |
| Compounds | cells | cells |
| ATRA | 7.6 | 10.85 |
| CI-994 | 0.29 | 0.17 |
| MS-275 | 0.19 | 0.009 |
| Sodium butyrate (BA) | 72.44 | >1000 |
| VNLG/60 (10) (AC linker)^{b} | 4.27 | 0.63 |
| VNLG/66 (13) (AC linker)^{b} | 0.04 | 0.94 |
| VNLG/114 (17) (1,6-E linker)^{b} | 0.18 | 0.17 |
| VNLG/122 (18) (1,6-E linker)^{b} | 0.87 | 0.02 |
| VNLG/124 (19),ATRA-BA (1,6-E | 1.02 | 0.01 |
| linker)^{b} | | |

^{a}The GI₅₀ values were determined from dose-response curves (by nonlinear regression analysis using GraphPad Prism) compiled from at least three independent experiments, SEM < 10% and represent the compound concentration (μM) required to inhibit cell growth by 50%.
^{b}AC linker = acyloxymethylcarbamate linker and 1,6-E linker = 1,6-elimination linker.

The novel mutual prodrugs exhibited differential antiproliferative potencies in both MDA-MB-231 and PC-3 cell lines. VNLG/66 with a GI₅₀=40 nM was the most potent versus the PC-3 cells.

TABLE 3
GI₅₀ values obtained from dose-response
curves in other breast cancer cell lines.

| | GI₅₀ Values (μM)^{a} | | | | |
|---|---|---|---|---|---|
| | MCF- | | LTLT- | MCF- | MCF- |
| Compounds | 7 | LTLC | Ca | 7_{TAMR} | 7_{HOX-B7} |
| (VNLG/60) (AC linker) | 0.25 | n/d | n/d | 0.17 | 1.20 |
| VNLG/66 (AC linker) | 0.15 | 0.02 | 0.65 | 0.02 | 0.006 |
| VNLG/114 (1,6-E linker) | 0.06 | n/d | n/d | 0.21 | 0.72 |
| VNLG/122 (1,6-E linker) | 0.52 | n/d | n/d | 0.08 | 8.13 |

^{a}The GI₅₀ values were determined from dose-response curves (by nonlinear regression analysis using GraphPad Prism) compiled from at least three independent experiments, SEM < 10% and represent the compound concentration (μM) required to inhibit cell growth by 50%. n/d = not determined.

Given the potency of most of the mutual prodrugs of the present invention, in MDA-MB-231 and PC-3 cell lines, their effects on the growth of some known drug-resistant breast cancer cell lines, including MCF-7_{TAMR}, MCF-7_{Hox-B7}, LTLC and LTLT-Ca was tested compared to parental MCF-7 cells. As presented in Table 3, most of the mutual prodrugs tested, including VNLG/60, VNLG/66, VNLG/114, and VNLG/122 resulted in potent inhibition of these resistant cell lines, with GI₅₀ values in the low nanomolar range.

### C. Others

### 1. Chemistry

a. (Acyloxy)alkyl Carbamate Type of Mutual Prodrugs

(Acyloxy)alkyl ester linker has been used for acid containing drugs to prepare prodrugs and mutual prodrugs as this linker is very labile and found to be cleaved by esterase enzyme. Mutual prodrug of ATRA and butyric acid (RN1) have been prepared using this concept. (Abraham Nudelman and Ada Rephaeli Novel Mutual Prodrug of Retinoic and Butyric Acids with Enhanced Anticancer Activity. J. Med. Chem. 2000, 43, 2962-2966; Nudelman, A.; Shaklai, M.; Aviram, A.; Rabizadeh, E.; Zimra, Y.; Ruse, M.; Rephaeli, A. Novel anticancer prodrugs of butyric acid. J. Med. Chem. 1992, 35, 6876-6894; Rephaeli, A.; Shaklai, M.; Ruse, M.; Nudelman, A. Derivatives of butyric acid as potential anti-neoplastic agents. Int. J. Cancer. 1991, 49, 66-72.). Amino functional drugs have been converted in the past to (acyloxy)alkyl carbamates (Jose Alexander, Robyn Cargill, Stuart R. Michelson, Harvey Schwam (Acyloxy)alkyl carbamates as novel bioreversible prodrugs for amines: increased permeation through biological membranes J. Med. Chem.; 1988; 31(2); 318-322; Jose Alexander, Robert A. Fromtling, Judith A. Bland, Barbara A. Pelak, Evamarie C. Gilfillan (Acyloxy)alkyl carbamate prodrugs of norfloxacin J. Med. Chem.; 1991; 34(1); 78-81) and found to bioreversible prodrugs for amine containing drugs. Mechanism of enzymatic hydrolysis of this type prodrug into drug is well established.

In order to prepare mutual prodrugs using this strategy, we synthesized p-nitrophenyl retinoyloxymethyl carbonate in three steps (Scheme 1). P-Nitrophenol was reacted with chloromethyl chloroformate in chloroform in presence of pyridine as base to afford chloromethyl-p-nitrophenyl carbonate (5). Chloro group was exchanged to iodo using sodium iodide in acetone to afford iodomethyl p-nitrophenyl carbonate (6) that was further reacted with ATRA using silver carbonate as base in acetone to give p-nitrophenyl retinoyloxymethyl carbonate (VNLG/34-B (“7” below)).

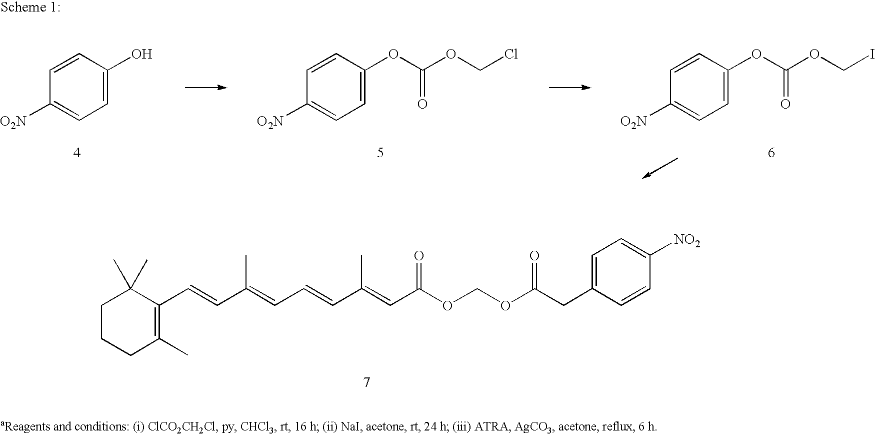

Attempt to condense aromatic amino group of CI-994 and MS-275 with this intermediate was unsuccessful. This could be due to low nucleophilicity of aromatic amine as well as steric hindrance as amino group is at ortho position. We decided to prepare amino acid derivative of CI-994 as this kind of derivatives have been used as prodrugs and they are borevesible (Nancy L. Pochopin, William N. Charman, and Valentino J. Stella. Pharmacokinetics of dapsone and amino acid prodrugs of dapsone. Drug Metab Dispos 1994 22: 770-775). Hence, glycine derivative of CI-994 was prepared in two steps. First we coupled N-Boc-glycine with CI-994 using DCC/HoBT in DMF and then deprotected with TFA in MDC to obtain glycine derivative of CI-994 (9). Reaction of this with p-nitrophenyl retinoyloxymethyl carbonate gave us the VNGL/60 (“10” below) (Scheme 2).

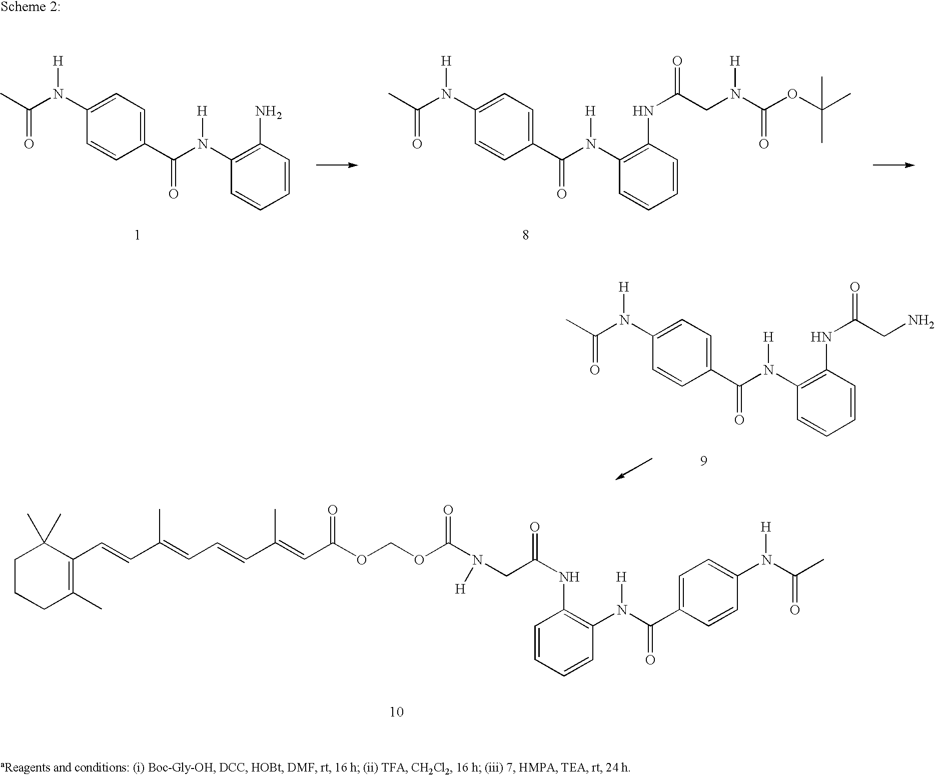

A similar synthetic scheme was used to synthesize mutual prodrug VNLG/66 (“13” shown below) of ATRA and MS-275 (Scheme 3).

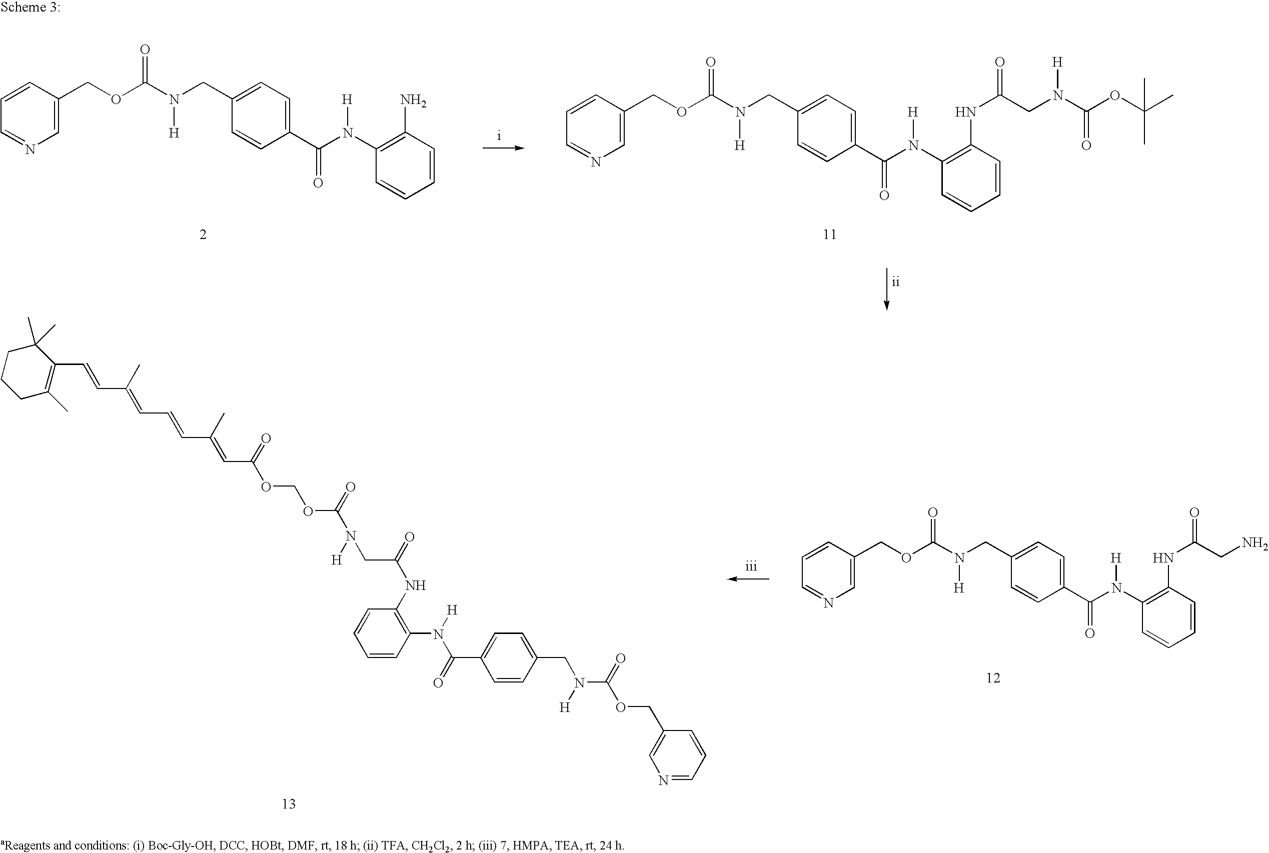

b. Elimination Based Prodrugs

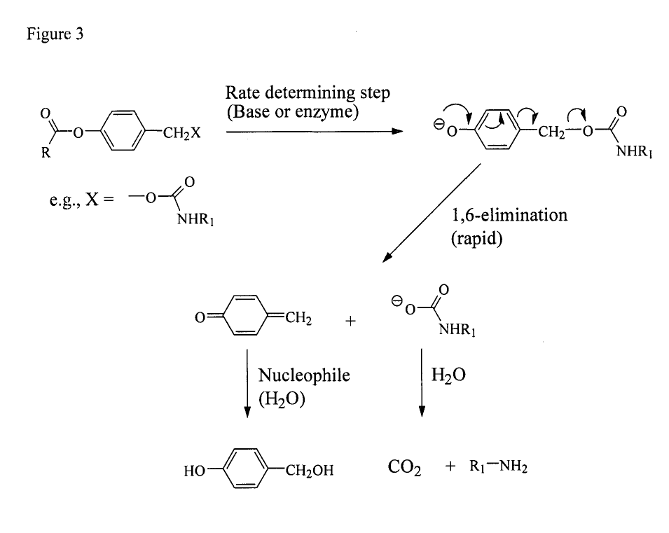

Generally, 1,4- or 1,6-elimination of HX (where X is a leaving group like a halide, functionalized oxygen derivatives such as carboxylates, or a carbamic acid anion) from benzyl compounds bearing strong electron-releasing o- or p-hydroxy or -amino substituents is a fast reaction that occurs under mildly basic conditions. Concomitantly, quinone methides and quinonimine methides are produced (Wakselman, M. 1,4- And 1,6-Eliminations From Hydroxy And Amino-Substituted Benzyl Systems: Chemical And Biochemical Applications. Nouv. J. Chem. 1983, 7, 439-447). A methodology which allows the use of these reactive compounds, especially for site-specific bioactivation, involves protecting the benzylic phenol (or aniline) derivative with a functionality that can be predictably hydrolyzed. This technology is generally referred to as the double-prodrug approach (Cain, B. F. 2-Acyloxymethylbenzoic Acids. Novel amine protective functions providing amides with the lability of Esters. J. Org. Chem. 1976, 41, 2029-2031; Bundgaard, H. The Double Prodrug Concept and Its Applications. Adv. Drug Delivery Rev. 1989, 3, 39-65). In such systems, the hydrolytic sequence involves a first step which usually is an enzymatic cleavage, followed by a second, faster step that is a molecular decomposition (FIG. 3). Although many prodrugs have been prepared using the 1,6-elimination concept especially for tumor targeted drug delivery (Florent, J. C.; Dong, X.; Gaudel, G.; Mitaku, S.; Monneret, C.; Gesson, J. P.; Jacquesy, J. C.; Mondon, M.; Renoux, B.; Andrianomenjanahary, S.; Michel, S.; Koch, M.; Tillequin, F.; Gerken, M.; Czech, J.; Straub, R.; Bosslet, K., Prodrugs of anthracyclines for use in antibody-directed enzyme prodrug therapy. J Med Chem 1998, 41, 3572-3581) there are very few examples of mutual prodrugs based on this concept. (Hulsman, N.; Medema, J. P.; Bos, C.; Jongejan, A.; Leurs, R.; Smit, M. J.; de Esch, I. J.; Richel, D.; Wijtmans, M., Chemical insights in the concept of hybrid drugs: the antitumor effect of nitric oxide-donating aspirin involves a quinone methide but not nitric oxide nor aspirin. J Med Chem 2007, 50, 2424-2431; Morphy, R.; Kay, C.; Rankovic, Z., From magic bullets to designed multiple ligands. Drug Discov Today 2004, 9, 641-651.)

First, all-trans-retinoic acid benzyl alcohol ester (11), a key intermediate to synthesize mutual prodrug of various HDACIs using 1,6 elimination concept (Scheme 4) was prepared. P-hydroxybenzaldehyde was coupled with ATRA using DCC/DMAP in DMF to yield benzaldehyde ester of all trans retinoic acid (10). Aldehyde was reduced to alcohol using sodium borohydride in isopropanol: chloroform (1:5) mixture to afford compound 16.

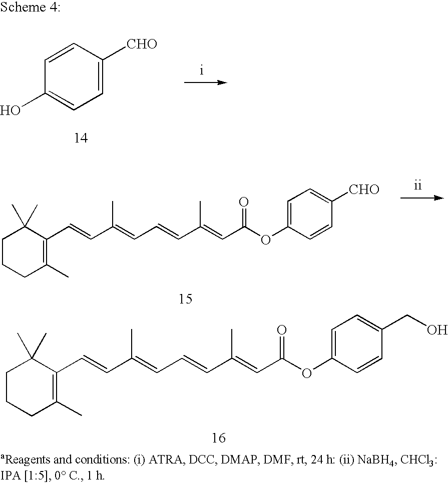

The chloroformate benzyl alcohol derivative was prepared using triphosgene. Initial attempt to prepare chloroformate using triphosgene/triethylamine yielded carbonate rather than chloroformate. Use of sodium carbonate in toluene afforded the desired chloroformate derivative. Chloroformate was used in next step without purification. Reaction of this chloroformate with MS-275 using pyridine as base in THF produced the desired mutual prodrug VNLG/114 (17 shown below). Similar reaction of chloroformate derivative with CI-994 gave mutual prodrug VNLG/122 (18 shown below). A reaction of alcohol (16) with butyric acid in the presence of DCC/DMAP yielded VNLG/124 (19 below).

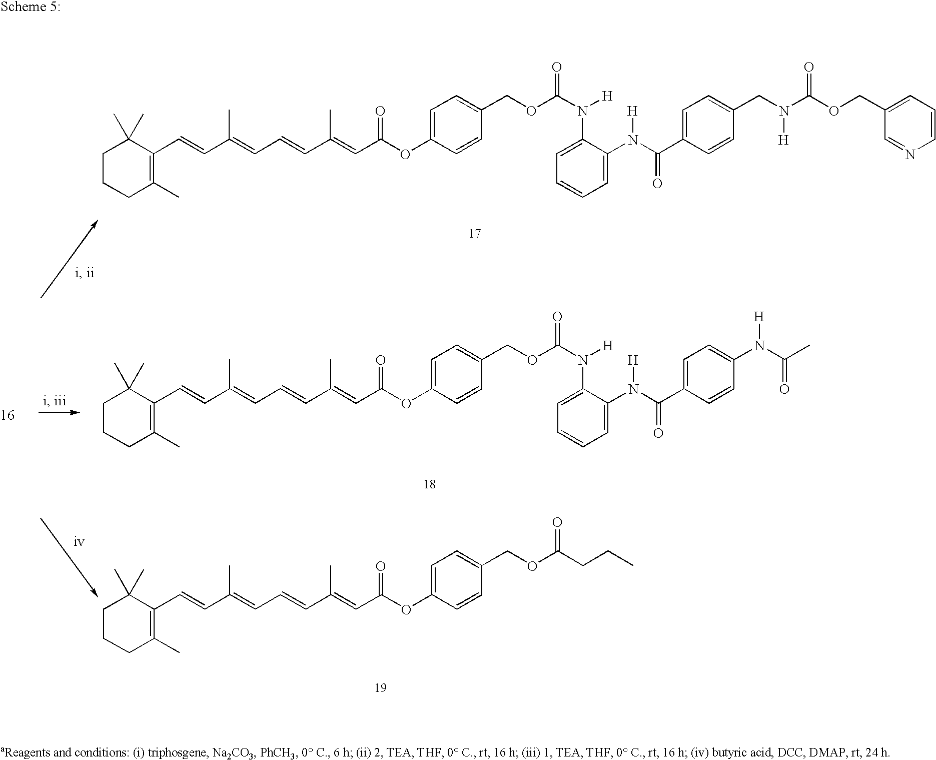

1. Experimental Section

a. Chemistry:

General procedures and techniques were identical with those previously reported. (Kelly, W. K.; Richon, V. M.; O'Connor, O.; Curley, T.; MacGregor-Curtelli, B.; Tong, W.; Klang, M.; Schwartz, L.; Richardson, S.; Rosa, E.; Drobnjak, M.; Cordon-Cordo, C.; Chiao, J. H.; Rifkind, R.; Marks, P. A.; Scher, H., Phase I clinical trial of histone deacetylase inhibitor: suberoylanilide hydroxamic acid administered intravenously. Clin Cancer Res 2003, 9, 3578-3588) ¹H NMR spectra were recorded in CDCl₃ and dmsod₆ at 500 MHz with Me₄Si as an internal standard using a Varian Inova 500 MHz spectrometer operating at 125 MHz or Bruker Advance DMX600 spectrometer operating at 150 MHz. High-resolution mass spectra (HRMS) were determined on a Karatos-Aspect Systems instrument, EI mode. Low-resolution mass spectra (LRMS) were determined on a Finnegan LCR-MS. Retinoids (all-trans-retinoic acid from LKT Laboratories, Inc., St. Paul, Minn. CI-994 and MS-275 were synthesized in our laboratory. All other precursors were purchased from Sigma-Aldrich.

Although the retinoidal intermediates and final products appeared to be relatively stable to light, precautions were taken to minimize exposure to any light source and to the atmosphere. Thus, all operations were performed in dim light, with reaction vessels wrapped with aluminum foil. All compounds were stored in an atmosphere of argon and in the cold (−20 or −80° C.) and dark without significant decomposition.

2-Chloromethyl-p-nitrophenyl carbonate (5): To an ice-cold mixture of p-nitrophenol (4, 1.39 g, 10 mmol) and pyridine (0.8 g, 10 mmol) in CHCl₃ (50 mL) was added chloromethyl chloroformate (1.41 g, 11 mmol). After approx. 30 min at 0-4° C., the reaction mixture was stirred further for 16 h at rt. Following successive washing with 0.5% aq. NaOH, and water, the CHCl₃ layer was dried over anhydrous NaSO₄ and evaporated to give thick yellow oil. This crude product was purified using flash column chromatography [FCC, Pet. ether/EtOAc, (9:1)] to obtain 5 (1.2 g, 52%). mp 44-45° C. ¹H NMR (CDCl₃): δ 5.85 (s, 2H, CH₂), 7.43 (d, 2H, J=7.5 Hz, Ar-Hs), 8.31 (d, 2H, J=9 Hz, Ar-Hs).

2-Iodomethyl-p-nitrophenyl carbonate (6): Compound 5 (2.0 g, 8.63 mmol) dissolved in acetone was treated with NaI (2.16 g, 14.42 mmol) and then stirred at rt for 24 h. The reaction mixture was evaporated and the residue was dissolved in CH₂Cl₂ followed by washing with saturated solution of sodium bisulfite and water. The organic layer was dried over anhydrous NaSO₄ and evaporated to obtain thick brown oil 6 (2.3 g). The crude product was used as such without further purification. ¹H NMR: δ 6.07 (s, 2H, CH₂), 7.43 (d, 2H, J=8.0 Hz, Ar-Hs), 8.31 (d, 2H, J=8.5 Hz, Ar-Hs).

(4-Nitrophenoxycarbonyloxy)methyl(2E,4E,6E,8E)-3,7-dimethyl-9-(2,6,6-trimethylcyclohex-1-enyl)nona-2,4,6,8-tetraenoate (7): ATRA (0.3 g, 1 mmol) was dissolved in acetone (15 mL) and to this was added Ag₂CO₃ (303 mg, 1.1 mmol) and refluxed for 1 h. The reaction mixture was cooled to rt (mixture A). Crude compound 6 (0.388 g) was dissolved in acetone (10 mL) separately and stirred at rt. Mixture A was added slowly to the solution of 6 followed by refluxing for 6 h. The reaction mixture was cooled to rt, filtered and the filtrate was evaporated to dryness. The crude product was purified by FCC [Pet. ether/EtOAc, (9.5:0.5)] to obtain pure 7 (129 mg, 26%): mp: 35-36° C. ¹H NMR (CDCl₃) δ 1.03 (s, 6H, 16 and 17-CH₃), 1.46 (m, 2H, CH₂), 1.60 (m, 2H, CH₂), 1.71 (s, 3H, 18-CH₃), 2.02 (s, 3H, 19-CH₃), 2.04 (m, 2H, CH₂), 2.40 (s, 3H, 20-CH₃), 5.82 (s, 1H, 14-H), 5.94 (s, 2H, CH₂), 6.23 (m, 4H, 7, 8, 10 and 12-Hs), 7.09 (dd, 1H, J=13.5 Hz, 11H), 7.42 (d, 2H, J=9.5 Hz, Ar-Hs), 8.29 (d, 2H, J=9 Hz, Ar-Hs).

N-(2-{[4-(Acetylamino)phenyl]carbonylamino}phenyl)-2-[(tert-butoxy)carbonylamino]acetamide (8): Boc-glycine (0.210 g, 1.2 mmol) and 1-hydroxybenzotriazole (HOBt) (0.162 g, 1.2 mmol) were dissolved in DMF (5 mL) and stirred at 0-5° C. To this added solution, was added 1 (0.269 g, 1 mmol) followed by dicyclohexylcarbodiimide (DCC) (0.248 g, 1.2 mmol). The cooling bath was removed after 30 minutes and the reaction mixture was stirred at rt for 18 h. The reaction mixture was poured into ice cold water and extracted with EtOAc. The organic layer was washed with brine, dried over anhydrous NaSO₄ and evaporated to dryness. The crude product was purified by FCC [CH₂Cl₂/EtOH, (9:1)] to afford 300 mg pure compound 8 (70%), mp: 125-126° C. ¹HNMR: δ 1.34 (s, 9H, CH₃), 2.08 (s, 3H, CH₃), 3.73 (s, 2H, CH₂) 7.21 (s, 2H, Ar-Hs), 7.59 (s, 1H, Ar—H), 7.63 (s, 1H, Ar—H), 7.72 (d, 2H, J=7.5 Hz, Ar-Hs), 7.94 (d, 2H, J=8.5 Hz, Ar-Hs), 9.51 (s, 1H, NH), 9.82 (s, 1H, NH), 10.22 (s, 1H, NH).

N-(2-{[4-(Acetylamino)phenyl]carbonylamino}phenyl)-2-aminoacetamide (9): To an ice cold solution of compound 8 (250 mg, 0.586 mmol) in CH₂Cl₂ (4 mL), was added TFA (4 mL) followed by stirring at 0-5 0° C. for 2 h. The reaction mixture was evaporated to dryness; acetone was added and stirred for 30 min. The white precipitate that formed was filtered and dried under vacuum to give pure compound 9 (148 mg, 77%), mp:215-218° C. ¹H NM R: δ 2.13 (s, 3H, CH₃), 3.42 (s, 1H, NH₂), 3.86 (s, 2H, CH₂), 7.28 (s, 1H, Ar—H), 7.68 (s, 1H, Ar—H), 7.77 (d, 2H, J=9 Hz, Ar-Hs), 7.99 (d, 2H, J=9 Hz, Ar-Hs), 8.17 (s, 2H, Ar-Hs), 9.71 (s, 1H, NH), 9.80 (s, 1H, NH), 10.30 (s, 1H, NH).

(N-{[N-(2-{[4-(Acetylamino)phenyl]carbonylamino}phenyl)carbamoyl]methyl}carbamoyloxy)methyl(2E,4E,6E,8E)-3,7-dimethyl-9-(2,6,6-trimethylcyclohex-1-enyl)nona-2,4,6,8-tetraenoate (10) (VNLG/60): To the solution of compound 7 (50 mg, 0.101 mmol) in hexamethylphosphoramide (HMPA) (1 mL) was added compound 9 (49 mg, 0.15 mmol) and Et₃N (210 μl, 0.15 mmol) and reaction mixture was stirred at rt for 24 h. The reaction mixture was poured into ice cold water and extracted with CH₂Cl₂. The organic layer was dried with anhydrous NaSO₄ and evaporated to dryness. The crude product was purified using FCC [CH₂Cl₂/EtOH, (20:1)] to give pure 10 (42 mg, 60%), mp: 32-33° C. IR (CHCl₃): 3429, 1734, 1676, 1599, 1508, 1457, 1335, 1297, 1214, 1066, 986, 754, 668 cm⁻¹. ¹H NMR (DMSO d₆): δ 1.03 (s, 6H, 16,17-CH₃), 1.71 (s, 3H, 18-CH₃), 2.02 (s, 3H, 19-CH₃), 2.13 (s, 3H, CH₃), 2.40 (s, 3H, 20-CH₃), 3.90 (s, 2H, CH₂), 5.59 (s, 2H, CH₂), 5.80 (s, 1H, 14H), 6.28 (m, 4H, 7-, 8-, 10- and 12-Hs), 7.20 (dd, 1H, J=14.7 Hz, 11-H), 7.23 (s, 2H, Ar—H), 7.61 (s, 1H, Ar—H), 7.63 (s, 1H, 9Ar—H), 7.72 (d, 2H, J=7.5 Hz, Ar-Hs), 7.94 (d, 2H, J=8.5 Hz, Ar-Hs), 8.1 (s, 1H, NH), 9.817 (s, 1H, NH), 10.3 (s, 1H, NH). HRMS calcd 683.3439 (C₃₉H₄₆N₄O₇H³⁰ ), found 683.3448.

2-[(Tert-butoxy)carbonylamino]-N-{2-[(4-{[(3-pyridylmethoxy)carbonylamino]methyl}phenyl)carbonylamino]phenyl}acetamide (11): Boc-glycine (0.210 g, 1.2 mmol) and HOBt (0.162 g, 1.2 mmol) was dissolved in DMF (5 mL) and stirred at 0-5° C. To this was added 2 (0.376 g, 1 mmol) followed by DCC (0.247 g, 1.2 mmol). The cooling bath was removed after 30 minutes and reaction mixture was stirred at rt for 18 h. The reaction mixture was poured into ice-cold water and extracted with EtOAC. The organic layer was washed with brine and dried over anhydrous NaSO₄ and then evaporated to dryness. The crude product was purified by FCC [CH₂Cl₂/EtOH (9:1)] to afford 380 mg (71%) of pure compound 11 as low melting solid. ¹H NMR (CDCl₃): δ 1.23 (s, 9H, CH₃), 3.87 (s, 2H, CH₂), 4.39 (d, 2H, J=5.5, CH₂), 5.14 (s, 2H, CH₂), 7.15 (m, 2H, Ar-Hs), 7.29 (d, 2H, J=7.5 Hz, Ar-Hs), 7.61 (d, H, J=8 Hz, Ar-Hs), 7.72 (d, 1H, J=7 Hz, Ar-Hs), 7.79 (m, 1H, Ar—H), 7.83 (d, 2H, J=8.5 Hz, Ar-Hs), 8.52 (s, 1H, NH), 8.58 (s, 1H, NH₂), 8.92 (s, 1H, NH), 9.24 (s, 1H, NH).

2-Amino-N-{2-[(4{[(3-pyridylmethoxy)carbonylamino]methyl}phenyl)carbonylamino]phenyl}acetamide (12): To an ice cold solution of compound 11 (300 mg, 0.562 mmol) dissolved in CH₂Cl₂ (4 mL) was added TFA (4mL) and stirred at 0-5° C. for 2 h. The reaction mixture was evaporated to dryness and to this was added acetone followed by stirring for 30 minutes. The white precipitate was filtered and dried under vacuum to give pure 12 as low melting solid (169 mg, 69%). ¹H NMR (DMSO d₆): δ 3.82 (s, 2H, CH₂), 4.28 (d, 2H, J=6 Hz, CH₂), 5.11 (s, 2H, CH₂), 7.24 (d, 1H, J=7 Hz, Ar-Hs), 7.39 (d, 1H, J=8 Hz, Ar—H), 7.45 (m, 1H, Ar—H), 7.65 (s, H, Ar—H), 7.83 (s, 1H, Ar), 7.95 (d, 2H, J=8 Hz, Ar-Hs), 8.08 (s, 1H, Ar—H), 8.25 (s, 1H, Ar—H), 8.623 (s,1H, Ar—H), 8.563 (s,1H, Ar—H), 9.55 (s, 1H, NH), 9.77 (s, 1H, NH), 10.02 (s, 1H, NH).

{N-[N-{2-[4-{[3-Pyridylmethoxy)carbonyamino]methyl}phenyl)carbonylamino]phenyl}carbamoyl carbamoyloxy}methyl(2E,4E,6E,8E)-3,7-dimethyl-9-(2,6,6-trimethylcyclohex-1-enyl)nona-2,4,6,8-tetraenoate (13) (VNLG/66): To the solution of 7 (50 mg, 0.101 mmol) in HMPA, added 12 (64.98 mg, 0.15 mmol) and Et₃N (210 μl, 0.15 mmol) and reaction mixture was stirred at rt for 24 h. The reaction mixture was poured into ice-cold water and extracted with CH₂Cl₂. The organic layer was dried over anhydrous Na₂SO₄ and evaporated to dryness. The crude product was purified using FCC [CH₂Cl₂/EtOH, (20:1)] to yield compound 13 (60 mg, 65%), mp: 56-58° C. IR (CHCl₃): 3684, 1715, 1651, 1592, 1519, 1477, 1336, 1296, 1214, 1123, 988, 754, 668 cm⁻¹. ¹H NMR (300 MHz, CDCl₃): δ 1.02 (s, 6H, 16,17-CH₃), 1.47 (m, 2H, CH₂), 1.61 (m, 2H, CH₂), 1.70 (s, 3H, 18-CH₃), 1.99 (s, 3H, 19-CH₃), 2.29 (s, 3H, 20-CH₃), 4.0 (s, 2H, CH₂), 4.43 (s, 2H, CH₂), 5.16 (s, 2H, CH₂), 5.71 (s, 1H, 4-H), 5.58 (s, 2H, CH₂), 5.80 (s, 1H, 14-H), 5.94 (s, 2H, CH₂), 6.23 (m, 4H, 7-, 8-, 10- and 12-Hs), 7.09 (dd, 1H, J=14.7 Hz, 11-H), 7.33 (m, 4H, Ar-Hs), 7.63 (d, 2H, J=7 Hz, Ar-Hs), 7.72 (s, 1H, Ar—H), 7.87 (s, 2H, Ar-Hs), 8.11 (s, 1H, NH), 8.09 (s, 1H, NH), 8.63 (s, 1H, NH), 9.51 (s, 1H, NH), 9.81 (s, 1H, NH), 10.21 (s, 1H, NH). HRMS calcd 790.3810 (C₄₅H₅₁N₅O₈Na⁺), found 790.3810.

4-Formylphenyl(2E,4E,6E,8E)-3,7-dimethyl-9-(2,6,6-trimethylcyclohex-1-enyl)nona-2,4,6,8-tetraenoate (15): ATRA (0.6 g, 2 mmol), 4-hydroxybenzaldehyde (14) 0.293 g, (2.4 mmol) and DMAP (0.293 g, 2.4 mmol) were dissolved in dry DMF and to this solution was added DCC (0.5 g, 2.4 mmol) at 0-10° C. The reaction mixture was stirred for 24 h at rt. The reaction mixture was filtered, poured into ice cold water and extracted with CH₂Cl₂. The organic layer was dried over anhydrous Na₂SO₄ and evaporated to give a crude product that was purified by FCC [CH₂Cl₂/EtOH, 9.5:0.5] to give the desired pure 15 (0.37 g, 91%), mp: 118-119° C. ¹H NMR (CDCl₃) δ 1.04 (s, 6H, 16, 17-CH₃), 1.32 (m, 2H, CH₂), 1.72 (s, 3H, 18-CH₃), 1.90 (m, 2H, CH₂), 2.03 (s, 3H, 19-CH₃), 2.42 (s, 3H, 20-CH₃), 5.99 (s, 1H, 14-H), 6.27 (m, 4H, 7,8,10,12-Hs), 7.10 (dd, 1H, 11-H), 7.31 (d, 2H, J=8.5 Hz, Ar-Hs), 7.92 (d, 2H, J=8.5 Hz, Ar-Hs), 9.99 (s, 1H, CHO).

4-(Hydroxymethyl)phenyl(2E,4E,6E,8E)-3,7-dimethyl-9-(2,6,6-trimethylcyclohex-1-enyl)nona-2,4,6,8-tetraenoate (16): Compound 15 (0.35 g, 0.86 mmol) was dissolved in IPA:CHCl₃ (1:5, 50 mL)) and cooled to 0° C. NaBH₄ (0.037 g) was then added to this and reaction mixture was stirred for 1 h at 0° C. The reaction was quenched by addition of acetone (1 mL), evaporated to dryness and purified by FCC [CH₂Cl₂/EtOH, (9:1)] to give pure 16 (0.32 g, 78.8%).mp: 89-90° C. ¹H NMR (CDCl₃) δ 1.03 (s, 6H, 16, 17-CH₃), 1.48 (m, 2H, CH₂), 1.62 (m, 2H, CH₂), 1.72 (s, 3H, 18-CH₃), 2.02 (s, 3H, 19-CH₃), 2.40 (s, 3H, 20-CH₃), 4.69 (s, 2H, CH₂), 5.99 (s, 1H, 14-H), 6.26 (m, 4H, 7,8,10,12-Hs), 7.08 (dd, 1H, 11-H), 7.11 (d, 2H, J=8.5 Hz, Ar-Hs), 7.38 (d, 2H, J=8.0 Hz, Ar-Hs).

4-{[N-(2-{[4-({[(3-Pyridylmethyl)oxycarbonyl]methyl}amino)phenyl]carbonylamino}phenyl)carbamoyloxy]methyl}phenyl(2E,4E,6E,8E)-3,7-dimethyl-9-(2,6,6-trimethylcyclohex-1-enyl)nona-2,4,6,8-tetraenoate (17) (VNLG/114): To a solution of triphosgene (118 mg, 0.39 mmol) in toluene (10 mL) at 0° C. was added NaHCO₃ (42 mg, 0.39 mmol) and reaction mixture was stirred for an 1 h. Compound 16 (135.0 mg, 0.32 mmol) dissolved in dry toluene (5 mL) was added drop wise over 30 min and the resulting reaction mixture was further stirred at 0° C. for 16 h. The reaction mixture was filtered and filtrate was evaporated to obtain dark brown oil which was reconstituted in THF (5 mL). This THF solution was added to the solution of 2 (124 mg, 0.33 mmol) and TEA (55 μl 0.39 mmol) in THF (5 mL) at 0° C. and then stirred further at rt for 16 h. The reaction mixture was evaporated and purified by FCC [CH₂Cl₂/EtOH, 9:1] to give compound 17 (110 mg, 40%) mp: 128-130° C. IR (CHCl₃): 3306, 1725, 1694, 1555, 1458, 1324, 1259, 1213, 1129, 1073, 749 cm⁻¹. ¹H NMR (300 MHz, DMSO d₆): δ 1.02 (s, 6H, 16, 17-CH₃), 1.48 (m, 2H, CH₂), 1.62 (m, 2H, CH₂), 1.70 (s, 3H, 18-CH₃), 2.01 (s, 3H, 19-CH₃), 2.34 (s, 3H, 20-CH₃), 4.28 (d, 2H, J=6 Hz, CH₂), 5.09 (s, 2H, CH₂), 5.14 (s, 2H, CH₂), 6.04 (s, 1H, 14-H), 6.25 (m, 4H, 7,8,10,12-Hs), 6.537 (s,1H, Ar—H), 6.507 (s, 1H, Ar—H), 7.16 (m, 4H, 11-H and Ar-Hs), 7.39 (d, 2H, J=7 Hz, Ar-Hs), 7.44 (d, 2H, J=8.5 Hz, Ar-Hs), 7.53 (d, 1H, J=8 Hz, Ar-Hs), 7.61 (d, 1H, J=8 Hz, Ar-Hs), 7.76 (d, 1H, J=7 Hz, Ar-Hs), 7.90 (d, 2H, J=7.5 Hz, Ar-Hs), 7.95 (s, 1H, Ar—H), 8.31 (s, 1H, Ar—H), 8.53 (s, 1H, Ar—H), 8.59 (s, 1H, NH), 9.05 (s, 1H, NH), 9.78 (s, 1H, NH). HRMS calcd 809.3908 (C₄₉H₅₂N₄O₇H⁺), found 809.3898.

4-{[N-2-{[4-(Acetylamino)phenyl]carbonylamino}phenyl0carbamoyloxy]methyl}phenyl(2E,4E,6E,8E) -3,7-dimethyl-9-(2,6,6-trimethylcyclohex-1-enyl)nona-2,4,6,8-tetraenoate (18) (VNLG/122): To a solution of triphosgene (118 mg, 0.39 mmol) in dry toluene (10 mL) at 0° C. was added HaHCO₃ (42 mg, 0.39 mmol) and reaction mixture was stirred for 1 h. Compound 16 (135 mg, 0.32 mmol) dissolved in dry toluene (5 mL) was added drop wise over 30 min and the resulting reaction mixture was further stirred at 0° C. for 16 h. The reaction mixture was filtered and filtrate was evaporated to obtain dark brown oil which was reconstituted in THF (5 mL). This THF solution was added to the solution of 1 (89 mg, 0.33 mmol) and TEA (55 μl, 0.39 mmol) in THF (5 mL) at 0° C. and then stirred further at rt for 16 h. The reaction mixture was evaporated and purified by FCC [CH₂Cl₂/EtOH, 9:1] to give compound 18 (98 mg, 42%): mp. 123-124° C. IR (CHCl₃): 3310, 1718, 1654, 1600, 1508, 1312, 1215, 1125, 758 cm⁻¹. ¹H NMR (300 MHz, DMSO d₆): δ 1.02 (s, 6H, 16,17-CH₃), 1.45 (m, 2H, CH), 1.57 (m, 2H, CH₂), 1.70 (s, 3H, 18-CH₃), 2.01 (s, 3H, 19-CH₃), 2.08 (s, 3H, CH₃), 2.35 (s, 3H, 20-CH₃), 5.14 (s, 4H, CH₂), 6.09 (s, 1H, 14-H), 6.26 (m, 4H, 7-, 8-, 10- and 12-Hs), 6.54 (s, 1H, CH), 6.54 (s, 1H, CH), 7.11 (d, 2H, J=8 Hz, Ar-Hs), 7.17 (m, 2H, Ar-Hs), 7.43 (d, 2H, J=8 Hz, Ar-Hs), 7.51 (d, 1H, J=7.5 Hz, Ar-Hs), 7.60 (d, 1H, J=7.5 Hz, Ar-Hs), 7.91 (d, 2H, J=8.0 Hz, Ar-Hs), 8.31 (s, 2H, Ar), 9.03 (s, 1H, NH), 9.73 (s,1H, NH), 10.22 (s, 1H, NH). HRMS calcd 702.3537 (C₄₃H₄₇N₃O₆H⁺), found 702.3541.

4-(Butanoyloxymethyl)phenyl(2E,4E,6E,8E)-3,7-dimethyl-9-(2,6,6-trimethylcyclohex-1-enyl)nona-2,4,6,8-tetraenoate (19) (VNLG/124): To a solution of butyric acid (42 mg, 0.47 mmol) in DMF (5 mL), added compound 16 (200 mg, 0.47 mmol), DCC (108 mg, 0.52 mmol) and DMAP (63.75, 0.52 mmol) and reaction mixture was stirred at rt for 24 h. The reaction mixture was poured into ice-cold water (50 mL) and extracted with CH₂Cl₂ (25 mL×3), the organic layer was dried over anhydrous Na₂SO4 and evaporated to dryness. The crude product was purified by FCC [pet. Ether/EtOAc, (50:1)] pure 19 as a yellow oil (123 mg, 52%): mp 38-40° C. IR (CHCl₃): 1723, 1577, 1353, 1234, 1214, 1123, 966, 753 cm⁻¹. ¹H NMR (300 MHz, DMSO d₆): δ 0.934 (t, 3H, J=7. 11¹CH₂), 1.03 (s, 6H, 16,17-CH₃), 1.11 (m, 2H, CH₂), 1.53 (m, 2H, CH₂), 1.68 (m, 2H, 10¹-CH₂), 1.72 (s, 3H, 18-CH₃), 2.02 (s, 3H, 19-CH₃), 2.33 (s, 3H, 20-CH₃), 2.46 (m, 2H, 9¹-CH₂), 5.10 (s, 2H, CH₂), 6.00 (s, 1H, 14-H), 6.27 (m, 5H, 7-, 8-, 10-,11 and 12-Hs), 7.11 (d, 2H, J=8 Hz, Ar-Hs), 7.37 (d, 2H, J=8.5, Ar). HRMS calcd 499.2818 (C₃₁H₄₀O₄Na⁺), found 499.2822.

### Kinetics of Hydrolysis in Plasma:

Plasma was obtained by centrifugation of blood samples containing 0.25% heparin. 0.8 ml of plasma was mixed with 0.2 ml of phosphate buffer (pH 7.2). Incubations were performed at 37° C.±0.5° C. using shaking water bath. The reaction was initiated by adding 25 μl of stock solution of mutual prodrugs (1 mg/ml) to preheated plasma and aliquots were taken at 3 and 6 hrs. Plasma samples underwent solid-phase extraction using 3 ml C18 Bond Elut columns (Varian, Harbor City, Calif.), which had previously been rinsed with 3 ml of methanol and 3 ml of distilled water. 500 μl of the sample were loaded, the column was washed with 3 ml of distilled water, and the drug was eluted with 2 ml of acetonitrile (In case of Mutual prodrugs of CI-994 1:1 Methanol and acetonitrile was used). Eluates were evaporated to dryness. Samples were then reconstituted in 400 μl of acetonitrile and filtered through a 0.45 μm filter (Ultrafree-MC; Millipore Corporation, Bedford, Mass.) before injection. Chromatographic analysis was achieved by a reverse-phase HPLC method on a Waters Novapak C18 column (3.9 mm×150 mm) protected by Waters guard cartridge packed with pellicle C18 as previously described. Briefly, the HPLC system used in this study consisted of a Waters solvent delivery system, a Waters controller (Milford, Mass.) coupled to a Waters 717plus autosampler, and a Waters 996 photodiode array detector operating at 240.0 nm and 350 nm. A multi-linear gradient solvent system, (i) 20 mM aqueous ammonium acetate buffer/methanol (50:50) (100% to 0%) and (ii) methanol (100%) (0% to 100%) at a flow rate of 0.8 ml/min, was used. Retention time for ATRA was 21.852 min, for MS-275 was 3.037 min and mutual Prodrug 13 was 25.634 min. HPLC analysis showed no mutual Prodrug after one hour where as ATRA and MS-275 were detected at 350 and 245 nm.

To be effective mutual prodrugs mutual prodrugs, the compounds of the present must revert rapidly and quantitatively to their two drugs in animal tissues and cell cultures. HPLC methods were developed to briefly study the cleavage of compounds VNLG/60, VNLG/66, VNLG/114,VNLG/122 and VNLG/124, in fresh mouse plasma. It was observed that all mutual prodrugs were completely cleaved to their parent compounds within 1 h of incubations at 37° C. Furthermore, the stability of mutual prodrugs was studied in 0.02 M phosphate buffer (pH=7.2) and also in 80% human serum (obtained from Sigma) containing 20% 0.02 M phosphate buffer as previously described.⁴¹ The mutual prodrugs were each incubated at 37° C. for 24 h, extracted and analyzed by HPLC. All the mutual prodrugs were found to be stable under these conditions. The difference in stabilities of the mutual prodrugs in fresh mouse plasma and commercial human serum may be due to deactivation of certain enzymes such as esterases and peptidases required for cleavage in human serum.

Result: All the mutual prodrugs were hydrolyzed in mice plasma at one hour into individual drugs (ATRA and HDAC inhibitors) confirming that these mutual prodrugs are bioreversible.

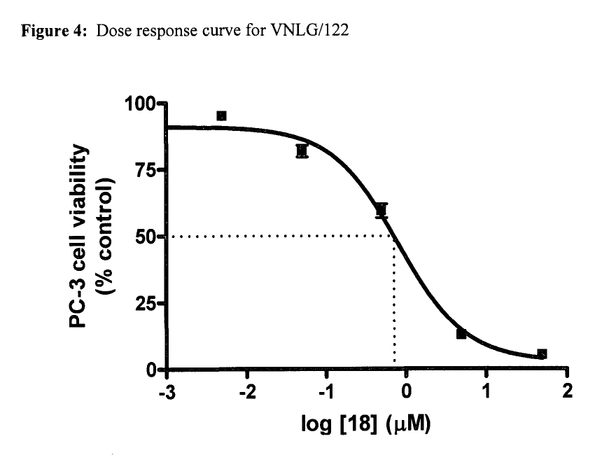

To assess the effect of mutual prodrugs on cell growth, PC-3 and MDA-MB-231 cells were treated with mutual prodrugs for 4 or days, respectively. A typical dose response curve for the antiproliferative effect of mutual prodrug VNLG/122 is presented in FIG. 4. Relative to other mutual prodrugs, VNLG/66 (ATRA-MS-275 with AC linker) was the most potent at inhibiting PC-3 cell growth (GI₅₀=40.0 nM) while VNLG/60 (ATRA-CI-994 with AC linker) was the least potent (GI₅₀=4.27 μM). In this cell line, the order of potency was VNLG/66>VNLG/114>VNLG/122>VNLG/124>VNLG/60. The efficacies of mutual prodrugs were compared to the efficacies of ATRA or HDIs alone in PC-3 prostate cancer cells. In general, the GI₅₀ values of all mutual prodrugs were 1.8- to 190-fold lower than that of ATRA and 17- to 1811-fold lower that of butyric acid (BA). Comparing the efficacies of mutual prodrugs with either of the HDIs, Intermediate compound 16 (ATRA-BA, with 1,6-elimination linker) exhibited the most benefit, since its GI₅₀ of 1.02 μM was 74-fold lower than BA. Given the potent cell growth inhibition (GI₅₀=190 nM) caused by compound 2, it is remarkable that mutual prodrug VNLG/66 (ATRA-MS-275 with AC linker) was still very potent with a GI₅₀ of 40 nM, 4.75-fold lower than 2. In contrast, mutual prodrugs with HDI 1 (CI-994), VNLG/60 and VNLG/122, with GI₅₀ values of 4.27 and 0.87 μM, respectively, were each less potent than HDI 1 (CI-994) (GI₅₀=0.29 μM). The reason(s) for these differential potencies of the different mutual prodrugs in PC-3 cells are unknown at this time, but may be idiosyncratic, possibly due to extents and efficiencies of cell membrane penetration and/or intracellular cleavage of mutual prodrugs.

Compared to their efficacies in PC-3 cells, the mutual prodrugs exhibited different potencies in the MDA-MB-231 cells. VNLG/66 (ATRA-MS-275 with AC linker) was the least potent (GI₅₀=940 nM). In this cell line, the order of potency was VNLG/124>VNLG/122>VNLG/114>VNLG/60>VNLG/66. Other notable observations on the antiproliferative effects of ATRA, HDIs and mutual prodrugs in this cell line were: (i) mutual prodrug VNLG/122 (ATRA-CI-994 with 1,6-E linker) with GI₅₀=20 nM is superior to related mutual prodrug VNLG/60 (ATRA-CI-994 with AC linker), GI₅₀=630 nM, a robust 31.5-fold difference, and (ii) VNLG/122 is also more potent than either ATRA (543-fold lower) or CI-994 (8.5-fold lower).

Some data suggests that the acyloxymethycarbamate linker is superior to the acyloxyalkyl linker.

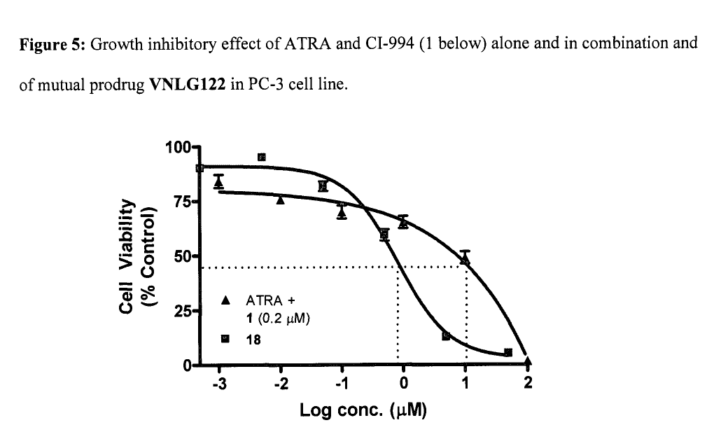

A comparison of mutual prodrugs VNLG/114 and VNLG/122 over simultaneous treatment of components of the mutual prodrugs were tested. The antiproliferative activities in both cell lines elicited by the MPs were observed to be each greater than those of the combined parent ATRA and HDIs (FIGS. 5, 6, and 8-10). Similar results were also obtained for VNLG/122 versus parent ATRA (increasing concentrations) and 1 (0.2 μM) (FIG. 5) and VNLG/114 versus parent ATRA and 2 (FIG. 6) in PC-3 cells.

### Butyric Acid

In addition to the mutual prodrugs of the present invention discussed above, the present application includes mutual prodrugs comprising the combination of the retinoic acids, retinoids, RAMBAs, etc. discussed above linked to butyric acid (BA). Butyric acid is a weak HDACI.

For example:

BA and ATRA.

BA and VN/66-1.

BA and VN/14-1.

BA and VN/12-1.

BA, 1,6 elimination linker and ATRA.

BA, 1,6 elimination linker and VN/66-1.

BA, 1,6 elimination linker and VN/14-1.

BA, 1,6 elimination linker and VN/12-1.

BA, acyloxymethylcarbamate linker and ATRA.

BA, acyloxymethylcarbamate linker and VN/66-1.

BA, acyloxymethylcarbamate linker and VN/14-1.

BA, acyloxymethylcarbamate linker and VN/12-1.

A non-limiting example is VNLG/124 (ATRA-BA (1,6-E linker)).

VNLG/124 is [4-(butanoyloxymethyl)phenyl(2E,4E,6E,8E)-3,7-dimethyl-9-(2,6,6-trimethylcyclohex-1-enyl)nona-2,4,6,8-tetraenoate].

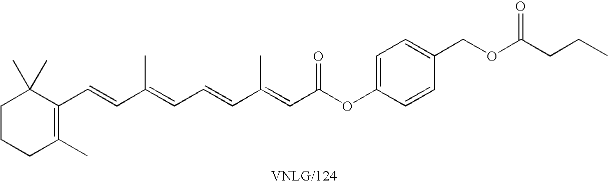

Relative to the mutual prodrugs of the present invention, VNLG/124 (19) (ATRA-BA with 1,6-elimination linker) was the most potent at inhibiting MDA-MB-231 cell growth (GI₅₀=10.0 nM).

Again, in PC-3 cell growth, the order of potency was VNLG/66>VNLG/114>VNLG/122>VNLG/124>VNLG/60.

Again, in the cell line MDA-MB-231 cells, the order of potency was VNLG/124>VNLG/122>VNLG/114>VNLG/60>VNLG/66.

A notable observation on the antiproliferative effects of ATRA, HDIs and VNLG/124 was: That VNLG/124 (19) exhibited the most benefit because its GI₅₀ of 10 nM was remarkably 1085-fold lower that that of ATRA and over 100,000-fold lower than BA.

Indeed, this gain in function of VNLG/124 (19) in this cell line is by far superior to that previously reported by Nudelman and Rephaeli (Nudelman, A.; Rephaeli, A., Novel mutual prodrug of retinoic and butyric acids with enhanced anticancer activity. J Med Chem 2000, 43, 2962-2966) for retinoyloxymethyl butyrate ((3), an MP derived from ATRA and BA with an acyloxyalkyl linker) in myeloid leukemia cell line HL-60. It might be unexpected that the coupling of ATRA to BA would cause such a large increase in activity, considering the low potency of BA. The results may be explained by a combination of two factors: (i) the ATRA fragment of VNLG/124 (19) imparts lipophilicity and facilitates the penetration of BA to the cellular target site; and (ii) the intracellularly released ATRA and BA affect the cells synergistically.

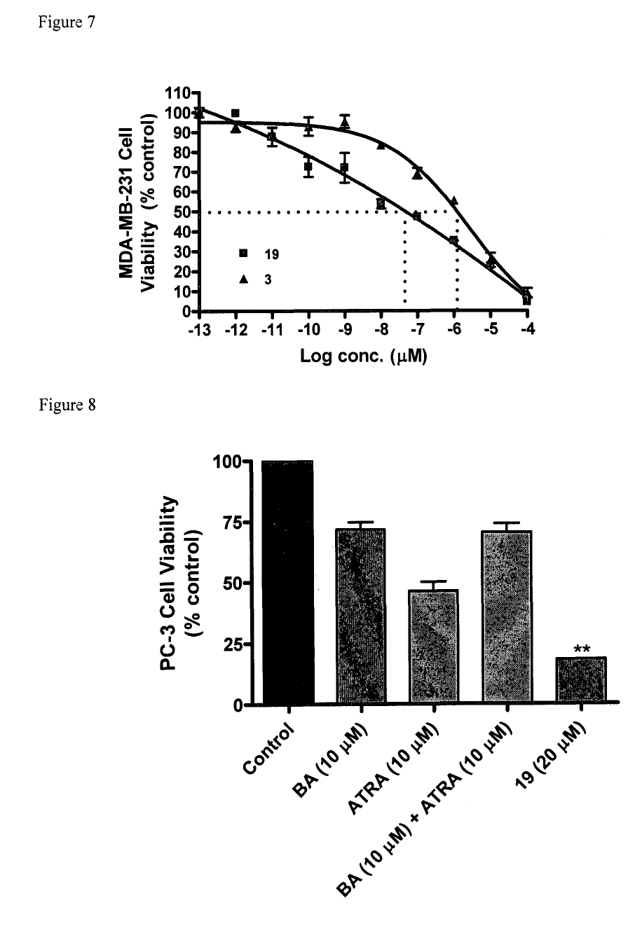

Furthermore, the efficacy of 3 was compared with that of our closely related VNLG/124 (19). Compound 3 was synthesized as previously described (Nudelman, A.; Rephaeli, A., Novel mutual prodrug of retinoic and butyric acids with enhanced anticancer activity. J Med Chem 2000, 43, 2962-2966) and assessed their antiproliferative activities head-to-head in MDA-MB-231 cells. As shown in FIG. 7, the GI₅₀ of VNLG/124 (19) for inhibition of growth of MDA-MB-231 cells was 48 nM, 25-fold lower than that of 3 (GI₅₀=1.18 μM). Together, these data suggest that the acyloxymethycarbamate linker is superior to the acyloxyalkyl linker. On the basis of the mean GI₅₀ values of all MPs obtained for the two cell lines, it tempting to suggest that that the 1,6 elimination linker with mean GI₅₀=0.035 μM (n=6) is superior to AC linker with mean GI₅₀=1.47 μM (n=4) (see Table 2). Validation of this assertion would probably require analysis of larger data set. It should be stated that some by-products resulting from intracellular cleavage of MPs, such as formaldehyde (generated from MPs with acyloxylalkyl linker) (Nudelman, A.; Rephaeli, A., Novel mutual prodrug of retinoic and butyric acids with enhanced anticancer activity. J Med Chem 2000, 43, 2962-2966; Nudelman, A.; Levovich, I.; Cutts, S. M.; Phillips, D. R.; Rephaeli, A., The role of intracellularly released formaldehyde and butyric acid in the anticancer activity of acyloxyalkyl esters. J Med Chem 2005, 48, 1042-1054) or quinine methide (generated from MPs with 1,6-elimination type linker) (Hulsman, N.; Medema, J. P.; Bos, C.; Jongejan, A.; Leurs, R.; Smit, M. J.; de Esch, I. J.; Richel, D.; Wijtmans, M., Chemical insights in the concept of hybrid drugs: the antitumor effect of nitric oxide-donating aspirin involves a quinone methide but not nitric oxide nor aspirin. J Med Chem 2007, 50, 2424-2431) have also been implicated in the anticancer activities of the two components of the MPs. Experiments to assess the possible involvement of by-products of our MPs are envisioned in future mechanistic studies.

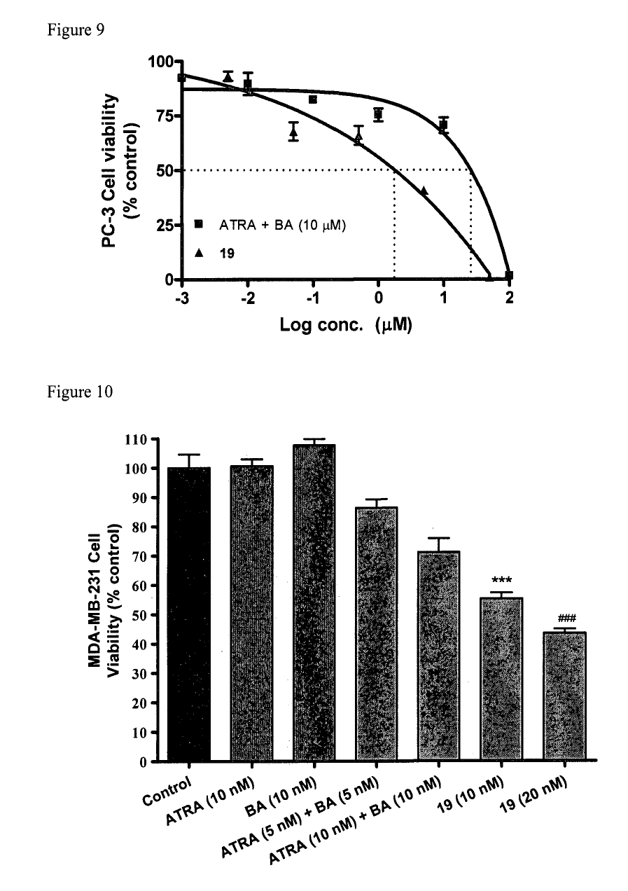

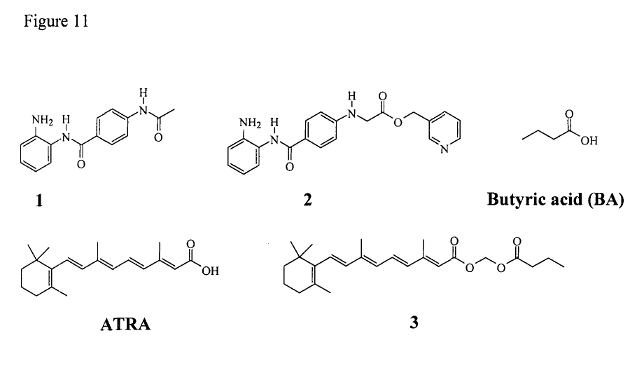

A comparison of VNLG/124 over simultaneous treatment of separate components of VNLG/124 was tested. The antiproliferative activities in both cell lines elicited by the MPs were observed to be each greater than those of the combined parent ATRA and HDIs (FIGS. 8-10). Treatment of PC-3 cells with 20 μM VNLG/124 (19) resulted in significantly potent growth inhibition (˜80%) compared to a mixture of 10 μM ATRA and 10 μM BA (FIG. 8). Furthermore, using dose-response curves, the antiproliferative activity elicited by VNLG/124 (19) (GI₅₀=1.7 μM) was 15-fold lower than the combination of increasing concentrations of ATRA and BA (10.0 μM) (GI₅₀=25.7 μM) (FIG. 9). Similar results were also obtained for VNLG/124 (19) versus parent ATRA and BA in MDA-MB-231 (FIG. 10).

In summary, with a GI₅₀ of 10 nM VNLG/124 was the most potent compound versus the MDA-MB-231 cells and VNLG/124 exhibited the most benefit because its GI₅₀ of 10 nM versus MDA-MB-231 cells was remarkably 1085-fold lower than that of parent ATRA and over 100,000-fold lower than butyric acid (BA).

While the invention has been described in detail and with reference to specific embodiments thereof, it will be apparent to one of ordinary skill in the art that various changes and modifications can be made therein without departing from the spirit and scope thereof The artisan will further acknowledge that the Examples recited herein are demonstrative only and are not meant to be limiting.

Each of the publications recited herein, including journal articles, books, manuals abstracts, posters, patents, and published patent applications, are hereby incorporated herein in their entireties.

1. A mutual prodrug compound comprising a histone deacetylase inhibitor (HDACI) linked to a retinoid.

2. The mutual prodrug composition of claim 1, wherein said HDACI is selected from the group consisting of SAHA, CI-994 and MS-275:

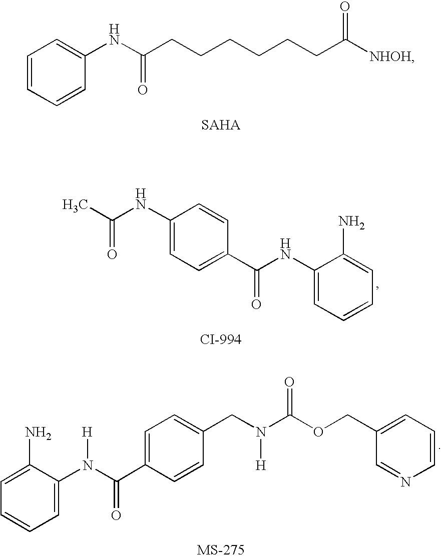

3. The mutual prodrug compound of claim 1, wherein said retinoid is selected from the group consisting of all-trans retinoic acid (ATRA) and 13-cis retinoic acid (13-CRA).

4. The mutual prodrug compound of claim 1, wherein said retinoid is selected from the group consisting of RAMBAs of formula (I): 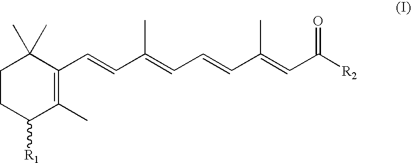
wherein
R₁ is an azole group, an allylic azole group, a sulfur-containing group, an oxygen-containing group, a nitrogen-containing group, a pyridyl group, an ethinyl group, a cyclopropyl-amine group, an ester group, an amino group, an azido group, or a cyano group, or R₁ forms, together with the C-4 carbon atom, an oxime, an oxirane or aziridine group;
R₂ is a hydroxyl group, an aminophenol group, an ester group, an azole group or —OR₃, wherein R₃ is selected from the group consisting of an alkyl, an aryl and a heterocyclic group; and
wherein, independently, any of the unsaturations may be cis or trans.

5. The mutual prodrug compound of claim 4, where said RAMBA selected from the group consisting of the RAMBAs set forth in Table 1:
TABLE 1

| Compound | R₁ | R₂ |
|---|---|---|
| VN/12-1^{t} | 1H-imidazole | —OCH₃ |
| VN/13-1^{t} | 1H-1,2,4-triazole | —OCH₃ |
| VN/13-2^{t} | 2H-1,2,4-triazole | —OCH₃ |
| VN/14-1^{t} | 1H-imidazole | —OH |
| VN/16-1^{t} | 1H-1,2,4-triazole | —OH |
| VN/17-1^{t} | 2H-1,2,4-triazole | —OH |
| VN/50A-1^{t} | 1H-imidazole | 1H-imidazole |
| VN/51A-1^{t} | Keto oxime | —OCH₃ |
| VN/66-1^{t} | 1H-imidazole | —NHC₆H₄OH |
| VN/65-4* | 1H-imidazole | —OCH₃ |
| VN/67-1* | 1H-imidazole | —OH |
| VN/68-1* | 1H-imidazole | 1H-imidazole |
| VN/69-1* | 1H-imidazole | —NHC₆H₄OH |

^{t}C-4 substituted ATRA analogs
*C-4 substituted 13-CRA analogs.

6. The mutual prodrug compound of claim 4, where said RAMBA selected from the group consisting of the VN/12-1, VN/14-1 and VN/16-1.

7. The mutual prodrug composition of claim 1, wherein said HDACI is CI-994 and said retinoid is all-trans retinoic acid (ATRA).

8. The mutual prodrug composition of claim 1, wherein said HDACI is MS-275 and said retinoid is all-trans retinoic acid (ATRA).

9. The mutual prodrug composition of claim 1, wherein said HDACI is linked to said retinoid via a linking group selected from the group consisting of an acyloxyalkyl linker, an (acyloxy)alkyl ester linker, an acyloxymethycarbamate linker, a glycine acyloxyalkyl carbamate linker a 1,6-elimination linker and a 1,4-elimination linker.

10. (canceled)

11. The mutual prodrug composition of claim 9, wherein said HDACI is linked to said retinoid via a linking group selected from the group consisting of a butyric acid and a phenyl butyric acid.

12. The mutual prodrug composition of claim 1, wherein said HDACI is linked to said retinoid via a linking group of the following formula: 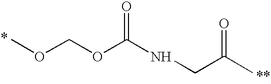
where * and ** are attachment points.

13. The mutual prodrug composition of claim 1, wherein said HDACI is linked to said retinoid via a linking group of the following formula: 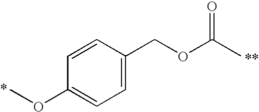
where * and ** are attachment points.

14. The mutual prodrug composition of claim 1, wherein said a histone deacetylase inhibitor (HDACI) linked to a retinoid is selected from the group consisting of:

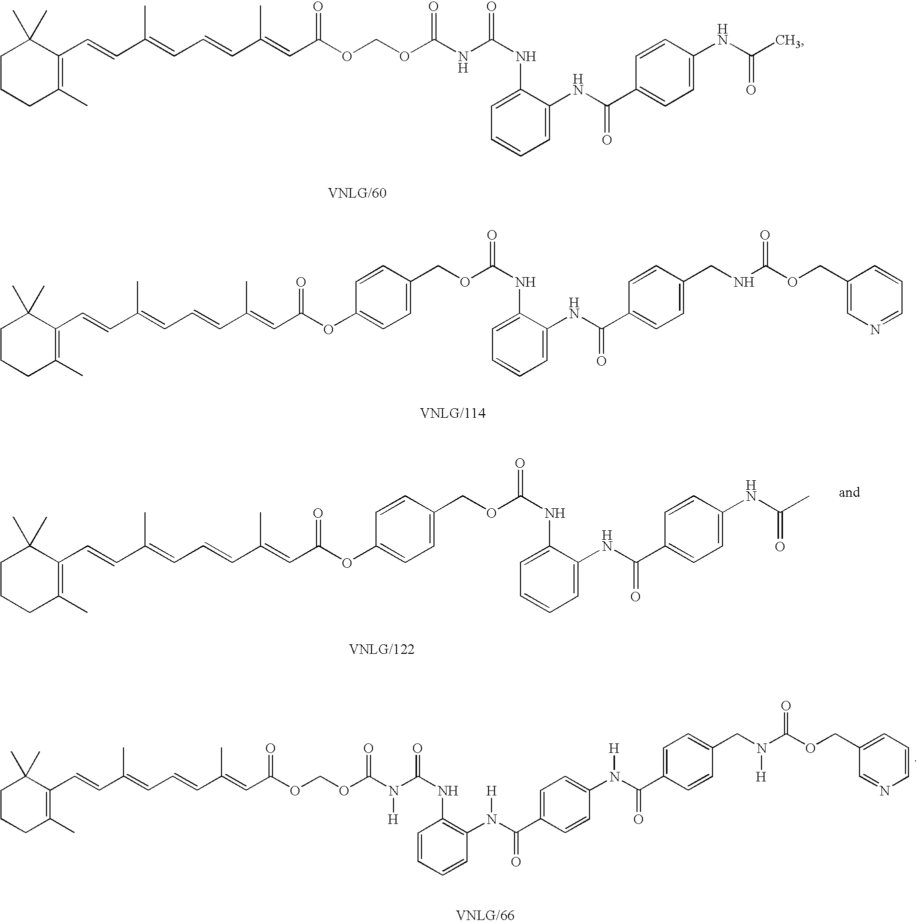

15. A pharmaceutically acceptable mutual prodrug composition comprising the mutual prodrug compound of claim 1 and a pharmaceutically acceptable carrier or an excipient.

16. A method of inhibiting ATRA 4-hydroxylase activity in a subject, comprising administering the pharmaceutically acceptable mutual prodrug composition of claim 15 to a subject, thereby inhibiting ATRA 4-hydroxylase activity in said subject.

17. A method of inhibiting growth of a cell in a subject, comprising administering the pharmaceutically acceptable mutual prodrug composition of claim 15 to a subject, thereby inhibiting the growth of a cell in said subject.

18. A method of treating cancer in a subject, comprising administering the pharmaceutically acceptable mutual prodrug composition of claim 15 to a subject in need of treatment, thereby treating cancer in said subject.

19. The method of claim 18, wherein said cancer is selected from the group consisting of an epithelial tumor, melanoma, leukemia, acute promyelocytic leukemia, lymphoma, osteogenic sarcoma, colon cancer, pancreatic cancer, breast cancer, prostate cancer, ovarian cancer, and lung cancer.

20. (canceled)

21. A compound of structural formula:

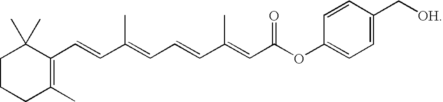

22. A mutual prodrug compound comprising a butyric acid linked to ATRA.

23. The mutual prodrug compound of claim 22, there the prodrug compound has formula

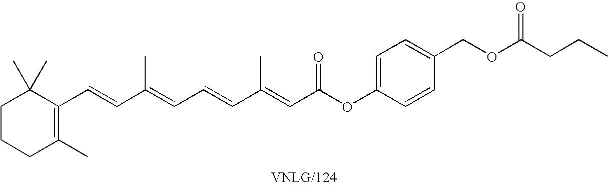

24. A pharmaceutically acceptable mutual prodrug composition comprising the mutual prodrug compound of claim 22 and a pharmaceutically acceptable carrier or an excipient.

25. A method of inhibiting ATRA 4-hydroxylase activity in a subject, comprising administering the pharmaceutically acceptable mutual prodrug composition of claim 24 to a subject, thereby inhibiting ATRA 4-hydroxylase activity in said subject.

26. A method of inhibiting growth of a cell in a subject, comprising administering the pharmaceutically acceptable mutual prodrug composition of claim 24 to a subject, thereby inhibiting the growth of a cell in said subject.

27. A method of treating cancer in a subject, comprising administering the pharmaceutically acceptable mutual prodrug composition of claim 24 to a subject in need of treatment, thereby treating cancer in said subject.

28. The method of claim 27, wherein said cancer is selected from the group consisting of an epithelial tumor, melanoma, leukemia, acute promyelocytic leukemia, lymphoma, osteogenic sarcoma, colon cancer, pancreatic cancer, breast cancer, prostate cancer, ovarian cancer, and lung cancer.

29. (canceled)
